(19) United States
(12) Patent Application Publication
(10) Pub. No.: US 2023/0194531 A1
(43) Pub. Date: Jun. 22, 2023

(54) Interferon Signaling Pathway-Related Gene Panel, Diagnostic Product and Application Thereof

(71) Applicant: Shanghai Precision Medicine Co., Ltd., Shanghai (CN)

(72) Inventors: Tong Zhou, Shanghai (CN); Jeff Weiqing Chou, Shanghai (CN); Zhiyuan Hu, Shanghai (CN); Linlin Ma, Shanghai (CN); Junhuan Lu, Shanghai (CN)

(21) Appl. No.: 17/905,521

(22) PCT Filed: Mar. 3, 2021
(86) PCT No.: PCT/CN2021/078805

§ 371 Date: Feb. 13, 2023

(30) Foreign Application Priority Data
Mar. 3, 2020 (CN) 202010141110.5

(51) Int. Cl. G01N 33/574

(52) U.S. Cl. CPC G01N 33/57415; G01N 33/57484

(57) ABSTRACT

The present disclosure relates to the field of biotechnologies. Specifically disclosed are an interferon signaling pathway-related gene panel and an in vitro diagnostic product thereof, and the use of the same in assessing the recurrence risk of breast cancer and/or providing guidance for breast cancer treatment with interferon.

The present application claims the priority of the Chinese Patent Application No. 202010141110.5, entitled “Interferon signaling pathway-related gene panel, diagnostic product and application thereof” filed on Mar. 3, 2020, of which the content is incorporated herein in its entirety by reference.

### TECHNICAL FIELD

The present disclosure relates to the field of biotechnology, and specifically relates to an interferon signaling pathway-related gene panel, a diagnostic product and application thereof.

### BACKGROUND

The incidence of breast cancer ranks first among the malignant tumors in women in China, and it is increasing at a rate of about 4% per year. Due to the high heterogeneity of breast cancer, there remains great challenge in precise treatment and reduction of recurrence risk after breast cancer surgery at home and abroad. Distant metastasis of breast cancer is the most serious type of breast cancer recurrence, which is an important indicator for prognosis, and the main cause of death in patients. Therefore, prediction of the risk of distant metastasis for breast cancer is particularly important for assessing and improving the prognosis of the patients. The molecular subtyping of breast cancer based on multi-gene expression profile in breast cancer tissue can categorize breast cancer into groups which reflecting tumor biology and may be used to assess the recurrence risk of each subtype, and providing guidance for chemotherapy, endocrine therapy or targeted therapy, which is of great guiding significance for clinical treatment.

With different molecular biological characteristics (e.g., expression of HER2, hormone receptor (ER/PR) or other tumor markers) and clinical and pathological indicators (e.g., patient age, tumor stage and presence of lymph nodes or the like), breast cancer differs greatly in the degree of malignancy, sensitivity to endocrine therapy, targeted therapy or chemotherapy, as well as prognosis. HER2-enriched and HER2-positive breast cancers are more sensitive to HER2-targeted therapy + chemotherapy, but the disease course progresses rapidly, and the prognosis is poor. Currently, it is desirable to develop detecting means and product for assessing the recurrence risk of HER2-enriched or HER2-posistive breast cancer and/or providing guidance for treatment of the same.

### SUMMARY

In an aspect, provided is a gene panel for assessing the recurrence risk of breast cancer and/or providing guidance for breast cancer treatment with interferon. Preferably, the breast cancer is HER2-enriched or HER2-posistive breast cancer. Preferably, the interferon is type I interferon.

In an embodiment, the gene panel according to the present disclosure comprises at least one of the following genes (G1): SAMD9, IFI35, IFIT3, OAS2, OASL and RTP4.

In a preferable embodiment, the gene panel according to the present disclosure comprises SAMD9 and at least one of the following genes (G1): IFI35, IFIT3, OAS2, OASL and RTP4.

In another embodiment, the gene panel according to the present disclosure comprises at least one of the following genes (G2): OAS3, DDX58, SP110, IFIH1, DDX60 and XAF1.

In another embodiment, the gene panel according to the present disclosure comprises at least one of the following genes (R): EIF2AK2, HERC5, HERC6, IFI27, IFI44, IFI44L, IFI6, IFIT1, IFIT5, IFITM1, ISG15, MX1, MX2, OAS1, PLSCR1, RSAD2 and USP18.

In a further embodiment, the gene panel according to the present disclosure comprises at least one of the following genes: SAMD9, IFI35, IFIT3, OAS2, OASL and RTP4, and/or at least one of the following genes: OAS3, DDX58, SP110, IFIH1, DDX60 and XAF1; and further comprises at least one of the following genes: EIF2AK2, HERC5, HERC6, IFI27, IFI44, IFI44L, FI6, IFIT1, IFIT5, IFITM1, ISG15, MX1, MX2, OAS1, PLSCR1, RSAD2 and USP18.

In a further preferable embodiment, the gene panel according to the present disclosure further comprises a reference gene(s). Preferably, the reference gene(s) comprises at least one, more preferably 3, most preferably 6 of: GAPDH, GUSB, MRPL19, PSMC4, SF3A1, TFRC, ACTB, RPLP0.

In a specific embodiment, the gene panel according to the present disclosure comprises: SAMD9, IFI35, IFIT3, OAS2, OASL and RTP4; as well as ACTB, GAPDH and RPLP0.

In another specific embodiment, the gene panel according to the present disclosure comprises: OAS3, DDX58, SP110, IFIH1, DDX60 and XAF1; as well as ACTB, GAPDH and RPLP0.

In yet another specific embodiment, the gene panel according to the present disclosure comprises: SAMD9, IFI35, IFIT3, OAS2, OASL, RTP4, OAS3, DDX58, SP110, IFIH1, DDX60, XAF1, EIF2AK2, HERC5, HERC6, IFI27, IFI44, IFI44L, IFI6, IFIT1, IFIT5, IFITM1, ISG15, MX1, MX2, OAS1, PLSCR1, RSAD2 and USP18; as well as GAPDH, GUSB, MRPL19, PSMC4, SF3A1 and TFRC.

In another aspect, provided is use of the gene panel according to the present disclosure in assessing the recurrence risk of breast cancer and/or providing guidance for breast cancer treatment with interferon.

In yet another aspect, provided is an agent for detecting the expression levels of the genes in the gene panel according to the present disclosure, for assessing the recurrence risk of breast cancer and/or providing guidance for breast cancer treatment with interferon.

In still another aspect, provided is a diagnostic product for assessing the recurrence risk of breast cancer and/or providing guidance for breast cancer treatment with interferon, comprising an agent for detecting the expression levels of the genes in the gene panel according to the present disclosure. In an embodiment, the diagnostic product is in a form of an in vitro diagnostic product. In a specific embodiment, the diagnostic product is in a form of a diagnostic kit. In a specific embodiment, the diagnostic product is a Next-Generation Sequencing kit, a Real-time fluorescence quantitative PCR detection kit, a gene chip, a protein microarray, an ELISA diagnostic kit or an Immunohistochemistry (IHC) kit.

In another aspect, provided is a method for assessing the recurrence risk of breast cancer and/or providing guidance for breast cancer treatment with interferon in a subject, comprising:
(1) providing a sample of the subject,
(2) determining the expression levels of the genes in the gene panel according to the present disclosure in the sample, optionally, calculating the interferon index according to the expression levels,
(3) judging the strength of the expression levels or the interferon index in (2),
(4) determining the recurrence risk of breast cancer and/or providing guidance for breast cancer treatment with interferon in the subject according to the expression levels or the interferon index in (3).

In another aspect, provided is use of the gene panel according to the present disclosure or the agent according to the present disclosure for the manufacture of a diagnostic product for assessing the recurrence risk of breast cancer and/or providing guidance for breast cancer treatment with interferon.

### BRIEF DESCRIPTION OF THE DRAWINGS

FIG. 1 shows effect of the strength of the interferon index calculated based on Gene panel G1 on the 10-year distant metastasis-free survival rate in HER2-enriched breast cancer cases subtyped according to the 72-gene panel for determining breast cancer molecular subtypes.

FIG. 2 shows effect of the strength of interferon index calculated based on Gene panel G1 on the 10-year distant metastasis-free survival rate in basal-like subtype, immune-enhanced subtype, Luminal A subtype and Luminal B subtype breast cancer cases subtyped according to the 72-gene panel for determining breast cancer molecular subtypes.

FIG. 3 shows effect of the strength of interferon index calculated based on Gene panel G2 or all 29 genes on the 10-year distant metastasis-free survival rate in HER2-enriched breast cancer cases subtyped according to the 72-gene panel for determining breast cancer molecular subtypes.

FIG. 4 shows effect of the strength of the expression level of each gene in Gene panel G1 on the 10-year distant metastasis-free survival rate in HER2-enriched breast cancer cases subtyped according to the 72-gene panel for determining breast cancer molecular subtypes.

FIG. 5 shows effect of the strength of the interferon index calculated based on Gene panel G1 on the 10-year distant metastasis-free survival rate in HER2-positive breast cancer cases (FIG. 5, left); and in HER2-negative breast cancer cases (FIG. 5, right).

FIG. 6 shows effect of the strength of the interferon index calculated based on Gene panel G1 on the 10-year distant metastasis-free survival rate in breast cancer cases which are HER2-positive and subtyped as HER2-enriched breast cancer according to the 72-gene panel for determining breast cancer molecular subtypes.

### DETAILED DESCRIPTION

### General Definition and Terms

The present disclosure will be described in details below, and it should be noted that the description is provided for the purposed of illustration rather than limitation.

Unless otherwise stated, the technical and scientific terms used herein have the same meaning as commonly understood by a person skilled in the art. If there is a contradiction, the definition provided in this application shall prevail. When a certain amount, concentration, or other value or parameter is set forth in the form of a range, a preferred range, or a preferred upper limit and a preferred lower limit, it should be understood that it is equivalent to specifically revealing any range formed by combining any upper limit or preferred value with any lower limit or preferred value, regardless of whether the said range is explicitly recited. Unless otherwise stated, the numerical ranges listed herein are intended to include the endpoints of the range and all integers and fractions (decimals) within the range.

Each reference cited herein (including all patents, patent applications, scientific publications, manufacturer’s instructions, guidelines, etc.) is incorporated by reference in its entirety. Nothing herein is to be construed as an admission that the invention is not entitled to antedate such disclosure by virtue of prior invention.

When used with a numerical variable, the term “approximate” or “about” usually refers to the value of the variable and all the values of the variable within the experimental error (for example, within the 95% confidence interval of the mean) or within ± 10% of the specified value, or a wider range.

The term “optional” or “optionally” means a subsequently described event or circumstance may or may not occur and that the description includes instances when the event or circumstance occurs and instances in which it does not. For example, when a group is described as optionally substituted, it can be unsubstituted or substituted, such as with one or more substituents independently selected from those described herein. It will be understood by those skilled in the art that the term also encompasses the meaning that the type and number of substituents are arbitrarily selected and combined, provided a stable compound is formed.

The expression “comprise” or its synonyms “contain”, “include”, “have” or the like are meant to be inclusive, which does not exclude other unlisted elements, steps or ingredients. The expression “consist of” excludes any unlisted elements, steps or ingredients. The expression “substantially consist of” refers to specified elements, steps or ingredients within a given range, together with optional elements, steps or ingredients which do not substantively affect the basic and novel feature of the claimed subject matter. It should be understood that the expression “comprise” encompasses the expressions “substantially consist of” and “consist of”.

The expression “and/or” covers cases of “and” and “or”. For example, “A and/or B” covers A, B as well as A and B. As another example, “A, and/or B, and/or C” can be understood in a similar manner, and can also be expressed as selecting at least one from A, B, C and any combination thereof, exemplarily covering A, B, C, A+B, A+C, B+C, A+B+C.

The expression “at least one” or the similar expression “one or more” refers to 1, 2, 3, 4, 5, 6, 7, 8, 9 or more. For example, when referring to containing at least one of the 17 genes in a gene panel, it can mean e.g., 1, 2, 3, 4, 5, 6, 7, 8, 9, 10, 11, 12, 13, 14, 15, 16 or 17.

“Breast cancer” originates from various levels of breast ducts and the acinar epithelium of the breast, and gradually develops from glandular epithelial hyperplasia to atypical hyperplasia. According to the degree of invasion of cancer cells to surrounding tissues and the possibility of distant metastasis, breast cancer can be categorized generally into carcinoma in situ (non-invasive carcinoma), early invasive carcinoma and invasive carcinoma. In a preferable embodiment, the breast cancer is an invasive breast cancer. Preferably, the breast cancer is HER2-enriched or HER2-positive breast cancer.

As used herein, the term “prognosis” refers to the prediction of the course and developmental outcome of breast cancer, including but not limited to the prediction of the probability of breast cancer recurrence, where the breast cancer with a lower probability of recurrence has a good prognosis, and while a higher probability means a poor prognosis.

As used herein, the term “recurrence” refers to the re-emergence of tumor cells after treatment within a specified observation period, and may include local recurrence, regional recurrence, or distant metastasis, depending on where the tumor cells reappear. As used herein, the term “recurrence” preferentially refers to distant metastasis or local recurrence of the breast cancer, more preferentially distant metastasis. As used herein, the term “local recurrence” refers to the recurrence of a tumor in the ipsilateral breast after breast conservation therapy for early breast cancer, or the recurrence of a tumor in the ipsilateral chest wall after mastectomy for operable breast cancer; “regional recurrence” refers to the presence of a tumor in the lymphatic drainage areas of the affected side, including axillary, supraclavicular/infraclavicular and internal mammary lymph node regions; “distant metastasis” refers to a tumor originating in the breast which has metastasized to distant organs or lymph nodes. The term “distant metastasis-free survival rate” as used herein refers to the proportion of breast cancer cases without distant metastasis during a specified observation period.

As used herein, the term “risk” refers to the probability or likelihood of an uncertain event occurring. Therefore, the likelihood of breast cancer recurrence can be expressed as “recurrence risk”, including but not limited to the risk of developing breast cancer local recurrence, regional recurrence, or distant metastasis. As used herein, the term “recurrence risk” preferentially refers to the risk of distant metastasis or local recurrence of breast cancer, and more preferentially that of distant metastasis, and can be represented by “distant metastasis-free survival rate”. Therefore, as used herein, a breast cancer with a higher distant metastasis-free survival rate has a lower risk of recurrence and a good prognosis; while a breast cancer with a lower distant metastasis-free survival rate has a higher risk of recurrence and a poor prognosis.

As used herein, the term “breast cancer molecular subtyping” refers to a breast cancer classification method established based on the gene expression profile of breast cancer tumor tissue.

Molecular subtyping systems for breast cancer that can be used include but are not limited to PAM50 (Prosigna) (see, for example, Parker, J. S. et al., Supervised risk predictor of breast cancer based on intrinsic subtypes. J. Clin. Oncol. 2009, 27: 1160-1167; or WO2009158143A1) and the 72-gene panel for determining breast cancer molecular subtypes (see, Yang B. et al., An assessment of prognostic immunity markers in breast cancer. NPJ breast cancer, 2018, 4: 35; or WO2020/064006A2. Unless otherwise specified, the specific 72-gene panel for determining breast cancer molecular subtypes used herein is the 72 gene molecular subtyping based on that disclosed in WO2020/064006A2 or Yang B. et al.) As an example, the PAM50 categorizes breast cancer into four subtypes as Luminal A, Luminal B, Basal-like, and HER2-enriched subtypes. As another example, the 72-gene panel for determining breast cancer molecular subtypes categorizes breast cancer into Luminal A, Luminal B, Basal-like, HER2-enriched and Immune-enhanced subtypes. In a preferable embodiment, the breast cancer molecular subtyping is performed using the 72-gene panel for determining breast cancer molecular subtypes.

As an example, the PAM50 molecular subtyping system (WO2009158143A1) categorizes breast cancer according to the expression profiles of 50 molecular subtyping-related genes, the 50 molecular subtyping-related genes comprise: ACTR3B, ANLN, BAG1, BCL2, BIRC5, BLVRA, CCNB1, CCNE1, CDC20, CDC6, CDCA1, CDH3, CENPF, CEP55, CXXC5, EGFR, ERBB2, ESR1, EXO1, FGFR4, FOXA1, FOXC1, GPR160, GRB7, HSPC150, KIF2C, KNTC2, KRT14, KRT17, KRT5, MAPT, MDM2, MELK, MIA, MKI67, MLPH, MMP11, MYBL2, MYC, NAT1, ORC6L, PGR, PHGDH, PTTG1, RRM2, SFRP1, SLC39A6, TMEM45B, TYMS and UBE2C. The PAM50 molecular subtyping system may further comprises a reference gene(s), e.g., MRPL19, PSMC4, SF3A1, PUM1, ACTB, GAPD, GUSB, RPLPO and TFRC, for normalizing and correcting the expression levels of the above 50 molecular subtyping-related genes. The PAM50 molecular subtyping diagnostic product comprises an agent for detecting the expression levels of the 50 molecular subtyping-related genes, and optional an agent for detecting the expression level(s) of a reference gene(s).

As an example, the 72-gene panel for determining breast cancer molecular subtypes may be that disclosed in Yang B. et al. For example, breast cancer is subtyped according to the expression profile of 66 molecular subtyping-related genes, the 66 molecular subtyping-related genes comprise: (1) 17 immune-related genes: APOBEC3G, CCL5, CCR2, CD2, CD27, CD3D, CD52, CORO1A, CXCL9, GZMA, GZMK, HLA-DMA, IL2RG, LCK, PRKCB, PTPRC and SH2D1A; (2) 14 estrogen receptor-related genes: BAG1, BCL2, BLVRA, CD68, ER, FOXA1, GSTM1, MAPT, MDM2, MLPH, NAT1, PGR, SCUBE2 and SLC39A6; (3) 19 proliferation-related genes: AURKA, BIRC5, CCNB1, CCNE1, CDC20, CDC6, CENPF, CEP55, EXO1, KIF2C, MELK, Ki67, MYBL2, NDC80, ORC6, PTTG1, RRM2, TYMS and UBE2C; (4) 11 basal cell-related genes: ACTR3B, CDH3, EGFR, FOXC1, KRT14, KRT17, KRT5, MIA, MYC, PHGDH and SFRP1; (5) 3 HER2-related genes: HER2, FGFR4 and GRB7; (6) 2 invasion-related genes: CTSL2 and MMP11. The 72-gene panel for determining breast cancer molecular subtypes may further comprise a reference gene(s), e.g., GAPDH, GUSB, MRPL19, PSMC4, SF3A1 and TFRC, for normalizing and correcting the expression levels of the above 66 molecular subtyping-related genes. The diagnostic product of the 72-gene panel for determining breast cancer molecular subtypes comprises an agent for detecting the expression levels of the 66 molecular subtyping-related genes described in Yang B. et al., and optionally an agent for detecting the expression level(s) of a reference gene(s).

As another example, the 72-gene panel for determining breast cancer molecular subtypes may be that disclosed in WO2020/064006A2. For example, breast cancer is subtyped according to the expression profile of 66 molecular subtyping-related genes, the 66 molecular subtyping-related genes comprise: (1) proliferation-related genes ASPM, AURKA, BIRC5, CCNB1, CDC20, CDK1, CENPU, CEP55, MELK, MKI67, NEK2, PRC1, PTTG1, RRM2, TOP2A, TPX2, TYMS, UBE2C and ZWINT; (2) immune-related genes APOBEC3G, CCL5, CCR2, CD2, CD3D, CD52, CD53, CORO1A, CXCL9, GZMA, GZMK, HLA-DMA, HLA-DQA1, IL2RG, LCK, LYZ and PTPRC; (3) basal cell-related genes ACTR3B, CDH3, EGFR, FOXC1, KRT14, KRT17, KRT5, MIA, MYC, PHGDH and SFRP1; (4) estrogen receptor-related genes BAG1, BCL2, BLVRA, CD68, ESR1, FOXA1, GSTM1, MAPT, MDM2, MLPH, NAT1, PGR, SCUBE2 and SLC39A6; (5) HER2-related genes ERBB2, FGFR4 and GRB7; (6) invasion-related genes CTSL2 and MMP11. The 72-gene panel for determining breast cancer molecular subtypes may further comprise a reference gene(s), e.g., GAPDH, GUSB, MRPL19, PSMC4, SF3A1 and TFRC, for normalizing and correcting the expression levels of the above 66 molecular subtyping-related genes. The diagnostic product of the 72-gene panel for determining breast cancer molecular subtypes comprises an agent for detecting the expression levels of the 66 molecular subtyping-related genes described in WO2020/064006A2, and optionally an agent for detecting the expression level(s) of a reference gene(s).

Human epidermal growth factor receptor 2 (HER2 protein) encoded by HER2/neu (also known as C-erbB2) gene is a member of the receptor tyrosine kinase family, which is an important protein regulating cell growth, proliferation and differentiation. The HER2 gene is amplified and/or overexpressed in various tumors, especially breast cancer and gastric cancer.

As used herein, the term “HER2-positive breast cancer” refers to the detection of amplification and/or overexpression of the HER2 gene using one or more methods, including gene amplification and/or overexpression detected at the nucleic acid or polypeptide level. For example, the overexpression of HER2 protein is detected by immunohistochemistry (IHC), the amplification of HER2 gene is detected by fluorescence in situ hybridization (FISH), and the high expression of HER2 mRNA is detected by Next-Generation Sequencing, but not limited to.

As used herein, the term “HER2-enriched breast cancer” refers to a breast cancer that is subtyped as HER2-enriched subtype through breast cancer molecular subtyping using, for example, PAM50 or the 72-gene panel for determining breast cancer molecular subtypes. In the above two subtyping systems, HER2-enriched breast cancer accounts for about 12% of all breast cancers with a poor prognosis.

As used herein, the HER2-positive breast cancer can be HER2-enriched or other molecular subtypes (e.g., Luminal A, Luminal B, Basal-like, Immune-enhanced); the HER2-enriched breast cancer can be HER2-positive, and a small part can also be HER2-negative.

As used herein, the term “interferons” are a group of cytokines with antiviral, growth inhibitory, and immunomodulatory effects stimulated by viruses or other inducements in the body. After action on the surface receptor of a target cell, interferons can activate the human immune system with a series of signaling which induce the expression of a variety of genes. Without bound by any mechanism, interferons can regulate the expression of a variety of genes related to the growth, proliferation, differentiation, migration or invasion of cancer cells. Interferons may comprise type I, type II and type III interferons. As used herein, the term “breast cancer treatment with interferon” refers to a treatment regimen applying one or more of the above-mentioned interferons in the clinical treatment of breast cancer, either alone or in combination with other treatment regimens (such as surgery, targeted therapy, chemotherapy, etc.). The term “guidance for breast cancer treatment with interferon” refers to the prediction of whether a breast cancer patient will benefit from a “breast cancer treatment with interferon” regimen. The term “interferon pathway signaling-related gene” refers to a gene whose expression level is regulated by interferon. As used herein, the interferon may be type I interferon.

The term “interferon index” herein refers to a weighted average index calculated according to the expression levels of genes related to the interferon signaling pathway of the present disclosure, which can be used to assess the recurrence risk in a patient with HER2-enriched or HER2-positive breast cancer. As used herein, according to the interferon index score, breast cancer can be categorized into two groups: strong or weak interferon index. A patient with HER2-enriched or HER2-positive breast cancer with a “strong” interferon index has a significantly lower recurrence risk than a patient with a “weak” interferon index. For a patient with a “weak” interferon index, it is possible to reduce the recurrence risk of breast cancer through combination with interferon therapy to enhance the interferon signaling pathway.

The term “polypeptide” herein refers to a compound composed of amino acids connected by peptide bonds, including a full-length polypeptide or an amino acid fragment. The term “target polypeptide” as used herein preferentially refers to the polypeptide, protein or protein fragment encoded by the gene to be detected.

The term “nucleotide” comprises deoxyribonucleotide and ribonucleotide. The term “nucleic acid” refers to a polymer composed of two or more nucleotides, encompassing deoxyribonucleic acid (DNA), ribonucleic acid (RNA) and nucleic acid analog. As used herein, the term “target nucleic acid” preferentially refers to the DNA, RNA transcript or cDNA complementary to the RNA transcript of the target gene. The term “RNA transcript” refers to total RNA, including coding or non-coding RNA, for example mRNA, rRNA or tRNA, directly derived from tissue or peripheral blood sample, or indirectly derived from tissue or blood sample after cell lysis. The term “mRNA” can include precursor mRNA and mature mRNA, either the full-length mRNA or its fragment. The RNA herein that can be used for detection is preferably mRNA, and more preferably mature mRNA. The term “cDNA” refers to DNA with a base sequence complementary to RNA. Those skilled in the art can apply methods known in the art to obtain the RNA transcript and/or cDNA complementary to its RNA transcript from the DNA of the gene, for example, by a chemical synthesis method or a molecular cloning method.

The term “hybridization” refers to the process of combining two nucleic acid fragments via stable and specific hydrogen bonds to form a double helix complex under appropriate conditions. The term “probe”, “hybridization probe” or “molecular probe” refers to a nucleic acid sequence (can be DNA or RNA) that includes at least 5 nucleotides, for example, 5-1000 nucleotides and can hybridize to a target nucleic acid or an amplified product thereof to form a complex under specific conditions. The term “TaqMan probe” is a probe based on TaqMan technology. Its 5′-end carries a fluorescent group, such as FAM, TET, HEX, NED, VIC or Cy5, etc., and its 3′-end carries a fluorescence quenching group (e.g., TAMRA and BHQ group) or non-fluorescence quenching group (TaqMan MGB probe). It has a nucleotide sequence that can hybridize to the target nucleic acid and can report the amount of the nucleic acid forming a complex with it when applied to Real-time fluorescence quantitative PCR (RT-PCR). The term “amplification primer” or “primer” refers to a nucleic acid fragment containing 5-100 nucleotides, preferably, 15-30 nucleotides capable of initiating an enzymatic reaction (e.g., an enzymatic amplification reaction).

The term “reference gene” herein refers to a gene that can be used as a reference to correct and normalize the expression level of the target gene. The reference gene inclusion criteria that can be considered are: (1) expression in tissues is stable, and the expression level is not affected by pathological condition or drug treatment or less affected; (2) the expression level should not be too high, to avoid a high proportion of the data acquired from the expression data (such as, those obtained through Next-Generation Sequencing), which will affect the accuracy of data detection and interpretation of other genes. Therefore, an agent that can be used to detect the expression level of the reference gene according to the present disclosure is also encompassed within the protection scope of the present disclosure.

The detection of gene expression level described herein can be done by detecting the amount of nucleic acid or polypeptide, with conventional technology in the art without any limitation. In the detection, the amount of the target polypeptide can be normalized against the amount of total protein, or the amount of the polypeptide encoded by the reference gene in the sample. In the detection, the amount of target nucleic acid, such as the DNA of the target gene, the RNA transcript or the amount of cDNA complementary to the RNA transcript, can be normalized against the amount of total DNA, total RNA or total cDNA, or the amount of DNA, RNA transcript or cDNA complementary to the RNA transcript of a group of reference genes in the sample.

### Gene Panel According to the Present Disclosure

In an aspect, provided is a gene panel for assessing the recurrence risk of breast cancer and/or providing guidance for breast cancer treatment with interferon. In some embodiments, the assessment of the recurrence risk of breast cancer and/or guidance for breast cancer treatment with interferon is provided based on the strength of the expression levels of the genes in the gene panel according to the present disclosure. In other embodiments, the assessment of the recurrence risk of breast cancer and/or the guidance for breast cancer treatment with interferon is provided based on the strength of the interferon index according to the present disclosure, wherein the interferon index is calculated based on the expression level of each gene in the gene panel according to the present disclosure and the respective contribution to the recurrence risk of breast cancer. The strength of the expression level of the gene or the strength of the interferon index are sufficient instructions for assessing the recurrence risk of breast cancer and/or providing guidance for breast cancer treatment with interferon for the subject. Preferably, the breast cancer is HER2-enriched or HER2-posistive breast cancer. In a preferable embodiment, the interferon is type I interferon.

In the embodiment according to the present disclosure, the gene panel comprises at least one gene in Gene panel G1, and/or at least one gene in Gene panel G2, and/or at least one gene in Gene panel R.

Gene panel G1 comprises the following genes: IFI35, IFIT3, OAS2, OASL, RTP4 and SAMD9 (also see information in Table 1).

Gene panel G2 comprises the following genes: OAS3, DDX58, SP110, IFIH1, DDX60 and XAF1 (also see information in Table 1).

Gene panel R comprises the following genes: EIF2AK2, HERC5, HERC6, IFI27, IFI44, IFI44L, IFI6, IFIT1, IFIT5, IFITM1, ISG15, MX1, MX2, OAS1, PLSCR1, RSAD2 and USP18 (also see information in Table 1).

Those skilled in the art should understand that the names of Gene panel G1, Gene panel G2, and Gene panel R are used only for grouping convenience and do not have specific denotative meaning. According to various embodiments, the gene panel according to the present disclosure may encompass one or more genes in Gene panel G1, and/or Gene panel G2, and/or Gene panel R, respectively, or any combination thereof, or it may encompass the entire Gene panel G1, and/or Gene panel G2, and/or Gene panel R. Those skilled in the art should also understand that the expression “at least one gene in Gene panel G1, and/or at least one gene in Gene panel G2, and/or at least one gene in Gene panel R” should be understood in a similar way, and it can also be expressed that for Gene panels G1, G2 and R, the embodiment of the gene panel according to the present disclosure can be selected from one or more of them, for example, G1, G2, R, G1 and G2, G1 and R, G2 and R, G1 and G2 and R. On this basis, in each case, at least one gene in G1, G2 and R is further and independently selected.

In an embodiment, the gene panel according to the present disclosure comprises at least one gene in Gene panel G1, for example, 1, 2, 3, 4, 5 or 6. In a preferable embodiment with Gene panel G1, the gene panel according to the present disclosure comprises SAMD9 and/or at least one gene of the following genes: IFI35, IFIT3, OAS2, OASL and RTP4. It should be understood that SAMD9 and/or at least one of (IFI35, IFIT3, OAS2, OASL and RTP4) belongs to a specific embodiment of Gene panel G1 (e.g., at least one gene). More preferably, the gene panel according to the present disclosure comprises SAMD9, IFI35, IFIT3, OAS2, OASL and RTP4.

In another embodiment, the gene panel according to the present disclosure comprises at least one gene in Gene panel G2, for example, 1, 2, 3, 4, 5 or 6. In a preferable embodiment, the gene panel according to the present disclosure comprises OAS3, DDX58, SP110, IFIH1, DDX60 and XAF1.

In a preferable embodiment, the gene panel according to the present disclosure comprises at least one gene in Gene panel G1 and at least one gene in Gene panel G2. In a preferable embodiment, the gene panel according to the present disclosure comprises SAMD9, and/or at least one of (IFI35, IFIT3, OAS2, OASL and RTP4), and/or at lest one gene in Gene panel G2. In a further embodiment, the gene panel according to the present disclosure comprises all the genes in Gene panel G1 and at least one gene in Gene panel G2. Alternatively, the gene panel according to the present disclosure comprises all the genes in Gene panel G2 and at least one gene in Gene panel G1. In a preferable embodiment, the gene panel according to the present disclosure comprises all the genes in Gene panel G1 and all the genes in Gene panel G2.

In another embodiment, the gene panel according to the present disclosure comprises at least one gene in Gene panel R, for example, 1, 2, 3, 4, 5, 6, 7, 8, 9, 10, 11, 12, 13, 14, 15, 16 or 17.

In another embodiment, the gene panel according to the present disclosure comprises at least one gene in Gene panel G1, and/or at least one gene in Gene panel G2, and at least one gene in Gene panel R. In a preferable embodiment, the gene panel according to the present disclosure comprises SAMD9, and/or at least one of (IFI35, IFIT3, OAS2, OASL and RTP4), and/or at least one gene in Gene panel G2, and at least one gene in Gene panel R.

In another preferable embodiment, the gene panel according to the present disclosure comprises all the genes in Gene panel G1, and at least one gene in Gene panel G2, and at least one gene in Gene panel R. Alternatively, the gene panel according to the present disclosure comprises all the genes in Gene panel G2, and at least one gene in Gene panel G1, and at least one gene in Gene panel R.

In a specific embodiment, the gene panel according to the present disclosure comprises all the genes in Gene panel G1, and/or all the genes in Gene panel G2, and/or all the genes in Gene panel R.

In an embodiment, in addition to the gene(s) in Gene panel G1 and/or Gene panel G2 and/or Gene panel R, the gene panel according to the present disclosure may further comprise a reference gene(s). Preferably, the reference gene(s) comprises at least one (e.g.,1, 2, 3, 4, 5, 6, 7 or 8), more preferably 3, most preferably 6 of: GAPDH, GUSB, MRPL19, PSMC4, SF3A1, TFRC, ACTB, RPLP0 (also see information in Table 1).

In an exemplary embodiment, the gene panel according to the present disclosure comprises at least one gene in Gene panel G1, and at least one of (ACTB, GAPDH and RPLPO).

In an exemplary embodiment, the gene panel according to the present disclosure comprises at least one gene in Gene panel G2, and at least one of (ACTB, GAPDH and RPLPO).

In another exemplary embodiment, the gene panel according to the present disclosure comprises at least one gene in Gene panel G1, at least one gene in Gene panel G2, and at least one of (GAPDH, GUSB, MRPL19, PSMC4, SF3A1 and TFRC).

In a preferable embodiment, the gene panel according to the present disclosure comprises SAMD9, IFI35, IFIT3, OAS2, OASL and RTP4; as well as ACTB, GAPDH and RPLP0.In an embodiment, the information of the gene panel according to the present disclosure can be found in Table 3.

In another preferable embodiment, the gene panel according to the present disclosure comprises: OAS3, DDX58, SP110, IFIH1, DDX60 and XAF1; as well as ACTB, GAPDH and RPLP0.

In another embodiment, the gene panel according to the present disclosure comprises: SAMD9, IFI35, IFIT3, OAS2, OASL, RTP4, OAS3, DDX58, SP110, IFIH1, DDX60, XAF1, EIF2AK2, HERC5, HERC6, IFI27, IFI44, IFI44L, IFI6, IFIT1, IFIT5, IFITM1, ISG15, MX1, MX2, OAS1, PLSCR1, RSAD2 and USP18; as well as GAPDH, GUSB, MRPL19, PSMC4, SF3A1 and TFRC. In an embodiment, the information of the gene panel according to the present disclosure can be found in Table 2.

TABLE 1

| Gene panel | No. | Gene name | Gene ID |
|---|---|---|---|
| G1 | 1 | SAMD9 | NM_001193307 |
| | 2 | RTP4 | NM_022147 |
| | 3 | OASL | NM_001261825 |
| | 4 | IFI35 | NM_005533 |
| | 5 | IFIT3 | NM_001031683 |
| | 6 | OAS2 | NM_001032731 |
| G2 | 7 | OAS3 | NM_006187 |
| | 8 | DDX58 | NM_014314 |
| | 9 | SP110 | NM_001185015 |
| | 10 | IFIH1 | NM_022168 |
| | 11 | DDX60 | NM_017631 |
| | 12 | XAF1 | NM_017523 |
| R | 13 | RSAD2 | NM_080657 |
| | 14 | HERC5 | NM_016323 |
| | 15 | MX2 | NM_002463 |
| | 16 | IFI44 | NM_006417 |
| | 17 | OAS1 | NM_001032409 |
| | 18 | IFIT5 | NM_012420 |
| | 19 | IFI44L | NM_006820 |
| | 20 | PLSCR1 | NM_021105 |
| | 21 | IFI27 | NM_001130080 |
| | 22 | MX1 | NM_001144925 |
| | 23 | IFI6 | NM_002038 |
| | 24 | HERC6 | NM_001165136 |
| | 25 | IFITM1 | NM_003641 |
| | 26 | EIF2AK2 | NM_001135651 |
| | 27 | ISG15 | NM_005101 |
| | 28 | IFIT1 | NM_001270927 |
| | 29 | USP18 | NM_017414 |
| Reference gene | 30 | GAPDH | NM_002046 |
| | 31 | GUSB | NM_000181 |
| | 32 | MRPL19 | NM_014763 |
| | 33 | PSMC4 | NM_006503 |
| | 34 | SF3A1 | NM_005877 |
| | 35 | TFRC | NM_003234 |
| | 36 | ACTB | NM_001101 |
| | 37 | RPLPO | NM_001002 |

TABLE 2

| No. | Function | Gene name | Gene ID |
|---|---|---|---|
| 1 | interferon pathway-related gene | DDX58 | NM_014314 |
| 2 | interferon pathway-related gene | DDX60 | NM_017631 |
| 3 | interferon pathway-related gene | EIF2AK2 | NM_001135651 |
| 4 | interferon pathway-related gene | HERC5 | NM_016323 |
| 5 | interferon pathway-related gene | HERC6 | NM_001165136 |
| 6 | interferon pathway-related gene | IFI27 | NM_001130080 |
| 7 | interferon pathway-related gene | IFI35 | NM_005533 |
| 8 | interferon pathway-related gene | IFI44 | NM_006417 |
| 9 | interferon pathway-related gene | IFI44L | NM_006820 |
| 10 | interferon pathway-related gene | IFI6 | NM_002038 |
| 11 | interferon pathway-related gene | IFIH1 | NM_022168 |
| 12 | interferon pathway-related gene | IFIT1 | NM_001270927 |
| 13 | interferon pathway-related gene | IFIT3 | NM_001031683 |
| 14 | interferon pathway-related gene | IFIT5 | NM_012420 |
| 15 | interferon pathway-related gene | IFITM1 | NM_003641 |
| 16 | interferon pathway-related gene | ISG15 | NM_005101 |
| 17 | interferon pathway-related gene | MX1 | NM_001144925 |
| 18 | interferon pathway-related gene | MX2 | NM_002463 |
| 19 | interferon pathway-related gene | OAS1 | NM_001032409 |
| 20 | interferon pathway-related gene | OAS2 | NM_001032731 |
| 21 | interferon pathway-related gene | OAS3 | NM_006187 |
| 22 | interferon pathway-related gene | OASL | NM_001261825 |
| 23 | interferon pathway-related gene | PLSCR1 | NM_021105 |
| 24 | interferon pathway-related gene | RSAD2 | NM_080657 |
| 25 | interferon pathway-related gene | RTP4 | NM_022147 |
| 26 | interferon pathway-related gene | SAMD9 | NM_001193307 |
| 27 | interferon pathway-related gene | SP110 | NM_001185015 |
| 28 | interferon pathway-related gene | USP18 | NM_017414 |
| 29 | interferon pathway-related gene | XAF1 | NM_017523 |
| 30 | reference gene | GAPDH | NM_002046 |
| 31 | reference gene | GUSB | NM_000181 |
| 32 | reference gene | MRPL19 | NM_014763 |
| 33 | reference gene | PSMC4 | NM_006503 |
| 34 | reference gene | SF3A1 | NM_005877 |
| 35 | reference gene | TFRC | NM_003234 |

TABLE 3

| No. | Function | Gene name | Gene ID |
|---|---|---|---|
| 1 | interferon pathway-related gene | IFI35 | NM_005533 |
| 2 | interferon pathway-related gene | IFIT3 | NM_001031683 |
| 3 | interferon pathway-related gene | OAS2 | NM_001032731 |
| 4 | interferon pathway-related gene | OASL | NM_001261825 |
| 5 | interferon pathway-related gene | RTP4 | NM_022147 |
| 6 | interferon pathway-related gene | SAMD9 | NM_001193307 |
| 7 | reference gene | ACTB | NM_001101 |
| 8 | reference gene | GAPDH | NM_002046 |
| 9 | reference gene | RPLPO | NM_001002 |

It should be understood by those skilled in the art that Table 1, Table 2 and Table 3 above and other similar tables below are provided for information only. Unless explicitly stated, it does not mean that all entries in the same table must be used together.

In specific embodiments, the gene panel according to the present disclosure is used for assessing the recurrence risk of breast cancer and/or providing guidance for breast cancer treatment with interferon. Preferably, the breast cancer is HER2-enriched or HER2-posistive breast cancer. In a preferable embodiment, the interferon is type I interferon.

The subject for who the gene panel according to the present disclosure is used for assessing the recurrence risk of breast cancer and/or providing guidance for breast cancer treatment with interferon can be the subject who has been assessed for HER2 or HER2-related gene status, for example, using one or more methods to detect amplification and/or overexpression of the HER2 gene in a sample of the subject, or subjected to breast cancer molecular subtyping using one or more breast cancer molecular subtyping systems. As an exemplary embodiment, the PAM50 or the 72-gene panel for determining breast cancer molecular subtypes (preferably the latter) can be used for assessment or subtyping. In a preferable embodiment, the subject is categorized as “HER2-positive breast cancer” or “HER2-enriched” breast cancer. More preferably, the categorization is performed with the PAM50 or the 72-gene panel for determining breast cancer molecular subtypes (preferably the latter).

### The Agent and Diagnostic Product According To The Present Disclosure

In another aspect, provided is an agent for detecting the expression levels of the genes in the gene panel according to the present disclosure and the use thereof for manufacture of a diagnostic product. The agent or diagnostic product can be used for assessing the recurrence risk of breast cancer and/or providing guidance for breast cancer treatment with interferon. Preferably, the breast cancer is HER2-enriched or HER2-posistive breast cancer. In a preferable embodiment, the interferon is type I interferon. The gene panel is described as above. It will be understood by those skilled in the art that selection of the agent or diagnostic products may each correspond to the gene in the gene panel according to the present disclosure. As an example, when multiple options are listed, such as primers of SEQ ID Nos: 1-70, it does not mean that the agent or diagnostic product according to the present disclosure should comprise all of these primers but that the agent or diagnostic product shall comprise those corresponding to the selected genes. This applies to the genes in the above Gene panel G1, and/or Gene panel G2, and/or Gene panel R, and/or reference genes.

In an alternative embodiment, the agent is an agent for detecting the amount of the polypeptide encoded by the gene. Preferably, the agent is an antibody, antibody fragment or affinity protein, which can specifically bind to the polypeptide encoded by the gene. More preferably, the agent is an antibody or antibody fragment capable of specifically binding to the polypeptide encoded by the gene. The antibody, antibody fragment or affinity protein may also have a label for detection, for example an enzyme (such as horseradish peroxidase), a radioisotope, a fluorescent label (such as Alexa Fluor dye, FITC, Texas Red, Cy3, Cy5, etc.), a chemiluminescent substance (such as luminol), biotin, a quantum dot (Qdot) or the like. Therefore, in a preferable embodiment, the agent is an antibody or antibody fragment capable of specifically binding to the polypeptide encoded by the gene, optionally having a label for detection, and the label is selected from an enzyme, a radioisotope, a fluorescent label, a chemiluminescent substance, biotin and quantum dot. In an embodiment, the agent is used to prepare a diagnostic product, which is a protein chip (such as a protein microarray), an ELISA diagnostic kit or an immunohistochemistry (IHC) kit.

In a preferable embodiment, the agent is an agent for detecting the amount of nucleic acid of the gene (e.g., DNA, RNA transcript or cDNA complementary to the RNA transcript of the gene), preferably an agent for detecting the amount of RNA transcribed from the gene, especially mRNA, or an agent for detecting the amount of cDNA complementary to the mRNA. Preferably, the agent is a probe or a primer or a combination thereof, wherein the probe or primer can be complementary to partial sequence of the gene in the gene panel according to the present disclosure, its RNA transcript, cDNA complementary to the RNA transcript and there is no limitation to the sequence. Preferably, there is high specificity. The probe or primer can be artificially synthesized.

In a preferable embodiment, the agent is a primer(s). In an embodiment, the primer(s) has a sequence as shown in SEQ ID NO. 1 - SEQ ID NO. 58, SEQ ID NO. 1 - SEQ ID NO. 70, SEQ ID NO. 71 - SEQ ID NO. 82 or SEQ ID NO. 71 - SEQ ID NO. 88.

In an embodiment, the agent is a probe(s). In an embodiment, the probe(s) has a sequence as shown in SEQ ID NO. 89 -SEQ ID NO. 94 or SEQ ID NO. 89 -SEQ ID NO. 97.

In an embodiment, the agent is a combination of a primer(s) and a probe(s). The primer(s) has a sequence as shown in SEQ ID NO. 1 - SEQ ID NO. 58, SEQ ID NO. 1 - SEQ ID NO. 70, SEQ ID NO. 71 - SEQ ID NO. 82 or SEQ ID NO. 71 - SEQ ID NO. 88. In an embodiment, the probe(s) has a sequence as shown in SEQ ID NO. 89 -SEQ ID NO. 94 or SEQ ID NO. 89 -SEQ ID NO. 97. In an embodiment, the primer(s) has a sequence as shown in SEQ ID NO. 71 - SEQ ID NO. 82 or SEQ ID NO. 71 - SEQ ID NO. 88, the probe(s) has a sequence as shown in SEQ ID NO. 89 -SEQ ID NO. 94 or SEQ ID NO. 89 -SEQ ID NO. 97.

In a specific embodiment, the primer(s) is used for quantitative PCR, including but not limited to semi-quantitative PCR and RT-PCR. In an embodiment, the primer(s) for quantitative PCR has a sequence as shown in SEQ ID NO. 71 - SEQ ID NO. 82 or SEQ ID NO. 71 - SEQ ID NO. 88 (also see Table 6). In another specific embodiment, the primer(s) is used for Next-Generation Sequencing, preferably for targeted sequencing. In a specific embodiment, the primer(s) is used for targeted sequencing and has a sequence as shown in SEQ ID NO. 1 - SEQ ID NO. 58 or SEQ ID NO. 1 - SEQ ID NO. 70 (also see Table 5). In an embodiment, the primers(s) is used for preparing a diagnostic product, which is a Next-Generation Sequencing kit based on targeted sequencing or a real-time fluorescence quantitative PCR kit.

In an embodiment, the agent is a probe(s), including but not limited to that for real-time fluorescence quantitative PCR (RT-PCR), in situ hybridization (ISH), southern blotting or northern blotting, gene chip technology or the like.

In a preferable embodiment, the probe(s) is used for RT-PCR. Preferably, the probe(s) has a sequence as shown in SEQ ID NO. 89 -SEQ ID NO. 94 or SEQ ID NO. 89 -SEQ ID NO. 97 (also see Table 6). Preferably, the probe(s) is a TaqMan probe(s). In a specific embodiment, the probe(s) is a TaqMan probe(s) having a sequence as shown in SEQ ID NO. 89 -SEQ ID NO. 94 or SEQ ID NO. 89-SEQ ID NO. 97. In an embodiment, the probe(s) is used for preparing a diagnostic product, which is a real-time fluorescence quantitative PCR detection kit.

In another preferable embodiment, the agent is a probe(s) and a primer(s) used for RT-PCR. In a preferable embodiment, the probe(s) is a TaqMan probe(s). In a more preferable embodiment, the probe(s) has a sequence as shown in SEQ ID NO. 89 -SEQ ID NO. 94 or SEQ ID NO. 89 -SEQ ID NO. 97 (also see Table 6). In a specific embodiment, the probe(s) is a TaqMan probe(s) having a sequence as shown in SEQ ID NO. 89 -SEQ ID NO. 94 or SEQ ID NO. 89 -SEQ ID NO. 97. In a preferable embodiment, the primer(s) has a sequence as shown in SEQ ID NO. 71 - SEQ ID NO. 82 or SEQ ID NO. 71 - SEQ ID NO. 88 (also see Table 6). In a more preferable embodiment, the probe(s) and primer(s) have sequences as shown in Table 6 (SEQ ID NO. 71-97). In a specific embodiment, the agent is a probe(s) and a primer(s) used for RT-PCR, wherein the probe(s) is a TaqMan probe(s) and has a sequence as shown in SEQ ID NO. 89 -SEQ ID NO. 94 (also see Table 6) and the primer(s) has a sequence as shown in SEQ ID NO. 71 - SEQ ID NO. 82 (also see Table 6). In a specific embodiment, the agent is a probe(s) and a primer(s) used for RT-PCR, wherein the probe(s) is a TaqMan probe and has a sequence as shown in SEQ ID NO. 89 -SEQ ID NO. 97 (also see Table 6), and the primer(s) has a sequence as shown in SEQ ID NO. 71 - SEQ ID NO. 88 (also see Table 6). In an embodiment, the probe(s) and primer(s) can be used to prepare a diagnostic product which is a real-time fluorescence quantitative PCR detection kit.

In an embodiment, the probe(s) is a probe that can be used for in situ hybridization, such as dual-color silver staining in situ hybridization (DISH), DNA fluorescence in situ hybridization (DNA-FISH), RNA fluorescence in situ hybridization (RNA-FISH), chromogenic in situ hybridization (CISH) or the like, the probe(s) can have a label, and the label can be a fluorescent group (such as Alexa Fluor dye, FITC, Texas Red, Cy3, Cy5, etc.), biotin, digoxin or the like. In another embodiment, the probe(s) can be used for gene chip detection, and the probe(s) can have a label which can be a fluorescent group. In a specific embodiment, the probe(s) can be used to prepare a diagnostic product which is a gene chip.

In another aspect, provided is a diagnostic product, which can be used for assessing the recurrence risk of breast cancer and/or providing guidance for breast cancer treatment with interferon. The product comprises an agent for detecting the expression levels of the genes in the gene panel according to the present disclosure. The gene panel is as described above. The agent is as described above. Preferably the breast cancer is HER2-enriched or HER2-posistive breast cancer. In a preferable embodiment, the interferon is type I interferon.

In an embodiment, the diagnostic product is in a form of an in vitro diagnostic product comprising the agent according to the present disclosure.

In a specific embodiment, the diagnostic product is in a form of a diagnostic kit comprising the agent according to the present disclosure.

In a specific embodiment, the diagnostic product can be a protein microarray, an ELISA diagnostic kit or an immunohistochemistry (IHC) kit, a Next-Generation Sequencing kit, a real-time fluorescence quantitative PCR kit, a gene chip or a combination thereof.

In a specific embodiment, the diagnostic product is a diagnostic product based on real-time fluorescence quantitative PCR, comprising the primer(s) and/or probe(s) described above. In another specific embodiment, the diagnostic product is a diagnostic product based on real-time fluorescence quantitative PCR, comprising the primer(s) and/or probe(s) having the nucleotide sequences as shown in Table 6.

In a preferable embodiment, the diagnostic product may further comprise at least one of: total RNA extraction reagent, reverse transcription reagent, Next-Generation Sequencing reagent, quantitative PCR reagent.

The total RNA extraction reagent is that conventionally used in the art. Examples include but are not limited to RNA storm CD201 (Cell Data Sciences), RNeasy FFPE Kit (Qiagen, #73504), PureLink RNA Mini Kit (Invitrogen).

The reverse transcription reagent can be that conventionally used in the art, and preferably comprises a dNTP solution and/or an RNA reverse transcriptase. Examples of reverse transcription reagents include but are not limited to ProtoScript® II reverse transcriptase (New England Biolabs, #M0368L) from NEB, RevertAid First Strand cDNA Synthesis Kit (ThermoFisher, #K1622) from ThermoFisher, TaqMan MicroRNA Reverse Transcription Kit (TaqMan™ MicroRNA Reverse Transcription Kit, Applied Biosystems, #4366596) from ABI.

The Next-Generation Sequencing reagent is that conventionally used in the art, as long as it can satisfy the requirements for Next-Generation Sequencing of the target nucleic acid. The Next-Generation Sequencing reagent can be commercially available, and examples include but are not limited to MiSeq® Reagent Kit (Illumina, #MS-102-3001) and TruSeq® Targeted RNA Index Kit A-96 Indices (Illumina, #RT-402-1001) from Illumina. The Next-generation sequencing technology is conventional in the art, preferably targeted RNA-seq. Therefore, the Next-Generation Sequencing reagent can also comprise Illumina-customized reagents for constructing a targeted RNA-seq library, such as TruSeq® Targeted RNA Custom Panel Kit (Illumina, #RT-102-1001) from Illumina.

The quantitative PCR reagent is that conventionally used in the art, as long as it can satisfy the requirements for quantitative PCR of the target nucleic acid. The quantitative PCR reagent is preferably commercially available. The quantitative PCR reagent is that conventionally used in the art, and preferably includes dNTPs solution and DNA polymerase. The quantitative PCR reagent is preferably that used for real-time quantitative PCR, such as that containing SYBR Green dye or that for TaqMan real-time quantitative PCR, more preferably that for TaqMan real-time quantitative PCR. The quantitative PCR reagent may optionally include a reagent for constructing a quantitative PCR library. The real-time PCR reaction can be performed by a PCR instrument for real-time fluorescence quantitative detection (e.g., ABI 7500 real-time fluorescence quantitative PCR instrument (Applied Biosystems) or LightCycler® 480II of Roche) where the gene expression levels are calculated.

In a specific embodiment, the diagnostic product is a Next-Generation Sequencing kit based on targeted RNA-seq, comprising primers having the nucleotide sequences shown in Table 5 and at least one selected from: total RNA extraction reagent, reverse transcription reagent, Next-Generation Sequencing reagent. The total RNA extraction reagent, reverse transcription reagent and Next-Generation Sequencing reagent are as described above. Preferably, the Next-Generation Sequencing reagent is Illumina-customized agent for constructing a targeted RNA-seq library.

In a specific embodiment, the diagnostic product is a PCR detection kit based on real-time fluorescence quantitative PCR, comprising primers and/or probes having the nucleotide sequences shown in Table 6 and at least one selected from: total RNA extraction reagent, reverse transcription reagent, quantitative PCR reagent. The total RNA extraction reagent, reverse transcription reagent and quantitative PCR reagent are as described above. Preferably, the quantitative PCR reagent is a real-time fluorescence quantitative PCR reagent.

Preferably, the diagnostic product according to the present disclosure (preferably in the form of a kit) further comprises a device for extracting a test sample from a subject; for example, a device for extracting the tissue or blood from a subject, preferably any blood collection needle, syringe or the like. The subject is a mammal, preferably a human, especially a patient with breast cancer, more preferably a HER2-enriched or HER2-positive breast cancer patient.

The subject suitable for the agent or diagnostic product according to the present disclosure can be that having been assessed for HER2 or HER2-related gene status, for example, using one or more methods to detect the amplification and/or overexpression of HER2 gene in a sample of the subject, or subjected to breast cancer molecular subtyping using one or more breast cancer molecular subtyping systems. As an exemplary embodiment, PAM50 or the 72-gene panel for determining breast cancer molecular subtypes (preferably the latter) can be used for assessment or subtyping. In a preferable embodiment, the subject is categorized as “HER2-positive breast cancer” or “HER2-enriched” breast cancer patient. More preferably, such categorization is performed with PAM50 or the 72-gene panel for determining breast cancer molecular subtypes (the latter is particularly preferable).

### The Method and Use According to the Present Disclosure

In another aspect, provided is also a method for determining the recurrence risk of breast cancer and/or providing guidance for breast cancer treatment with interferon in a subject, comprising,
(1) providing a sample of the subject,
(2) determining the expression levels of the genes in the gene panel according to the present disclosure in the sample, optionally, calculating the interferon index according to the expression levels,
(3) judging the strength of the expression levels or the interferon index in (2),
(4) determining the recurrence risk of breast cancer and/or providing guidance for breast cancer treatment with interferon in the subject according to the expression levels or the interferon index in (3).

The subject used in the method according to the present disclosure is a mammal, preferably a human, especially a breast cancer patient. The breast cancer is preferably HER2-enriched or HER2-positive breast cancer. The interferon can be type I interferon.

The sample used in step (1) is not particularly limited, as long as the expression levels of the genes in the gene panel can be obtained therefrom, for example, that from which the total genome RNA, total protein or the like of the subject can be extracted, preferably the total RNA. The sample is preferably a sample of tissue, blood, plasma, body fluid or a combination thereof, preferably a tissue sample, especially a paraffin tissue sample. In a preferable embodiment, the sample is a tumor tissue sample or a tissue sample containing tumor cells. In the embodiments according to the present disclosure, the sample may be that has been assessed for HER2 or HER2-related gene status, for example, using one or more methods to detect amplification and/or overexpression of the HER2 gene in the sample, or that has been subjected to molecularly subtyping for breast cancer using one or more breast cancer molecular subtyping systems. As an exemplary embodiment, PAM50 or the 72-gene panel for determining breast cancer molecular subtypes (preferably the latter), can be used for assessment or subtyping. In a preferable embodiment, the sample of the subject is categorized as “HER2-positive breast cancer” or “HER2-enriched” breast cancer sample. More preferably, such categorization is performed using PAM50 or the 72-gene panel for determining breast cancer molecular subtypes (the latter is particularly preferable).

In step (2), various methods can be used to determine the expression levels of the genes in the gene panel according to the present disclosure, including but not limited to determining the amounts of nucleic acids of the genes and the polypeptides encoded thereby. Those skilled in the art can select the type and amount of the sample in step (1) and select conventional technology in the art to achieve the determination in step (2).

In an embodiment, step (2) can be achieved by detecting the amounts of the polypeptides encoded by the genes. The detection can be achieved by the above-mentioned reagents and technology known in the art including but not limited to enzyme-linked immunosorbent assay (ELISA), chemiluminescence immunoassay technology (e.g., immunochemiluminescence assay, chemiluminescence enzyme immunoassay, electrochemiluminescence immunoassay), flow cytometry, immunohistochemistry (IHC).

In a preferable embodiment, step (2) can be achieved by detecting the amounts of nucleic acids of the genes. The detection can be achieved by the above-mentioned reagents and technology known in the filed including but not limited to molecular hybridization technology, quantitative PCR technology, nucleic acid sequencing technology or the like. Molecular hybridization technology includes but not limited to ISH technology (such as DISH, DNA-FISH, RNA-FISH, CISH technology, etc.), southern blotting or northern blotting technology, gene chip technology (such as microarray chip or microfluidic chip technology) or the like, wherein in situ hybridization technology is preferable. Quantitative PCR technology includes but not limited to semi-quantitative PCR and RT-PCR technology, preferably RT-PCR technology. Nucleic acid sequencing technology includes but not limited to Sanger sequencing, Next-Generation Sequencing (NGS), third-generation sequencing, single-cell sequencing technology or the like, preferably Next-Generation Sequencing, more preferably targeted RNA-seq technology.

In a preferable embodiment, in step (2), the expression levels of the genes in the gene panel according to the present disclosure is determined using the Next-Generation Sequencing technology. The gene panel is as described above, and reference can also be made to Table 2. In a specific embodiment, step (2) may comprise:
(2-1) extracting total RNA from the sample;
(2-2) converting the total RNA in step (2-1) into cDNA, which is then prepared into a library for Next-Generation Sequencing;
(2-3) subjecting the library obtained in step (2-2) to sequencing.

The extraction of step (2-1) can be performed by a conventional method in the art, preferably using a commercially available RNA extraction kit to extract total RNA from fresh frozen tissue or paraffin-embedded tissue of the subject.

In a preferable embodiment, step (2-2) may comprise the following: (i) reverse-transcribing the extracted total RNA to generate the cDNAs of the 35 genes as described in Table 2; (ii) preparing the obtained cDNAs into a library for sequencing.

Step (2-3) can be achieved by RNA sequencing. The primers in the kit are used to amplify the genes as shown in Table 2. Depending on the library prepared in step (2-2), the obtained genes can be subjected to Next-Generation Sequencing. Preferably, the Next-Generation Sequencing is targeted RNA-seq technology. The NextSeq/MiSeq/MiniSeq/iSeq sequencer from Illumina can be used for paired-end sequencing or single-end sequencing.

In another preferable embodiment, in step (2), RT-PCR method is used to determine the expression levels of the genes in the gene panel according to the present disclosure. The gene panel is as described above, and reference can also be made to Table 3. In a specific embodiment, step (2) can comprise:
(2-1) extracting total RNA from the sample;
(2-2) reverse transcribing the total RNA in (2-1) into cDNA;
(2-3) subjecting the cDNA to RT-PCR detection.

The extraction of step (2-1) can be performed by a conventional method in the art, preferably using a commercially available RNA extraction kit to extract total RNA from fresh frozen tissue or paraffin-embedded tissue of the subject.

The reverse transcription in step (2-2) can be performed using a commercially available reverse transcription kit.

In a preferable embodiment, the RT-PCR method in step (2-3) is TaqMan RT-PCR. The genes shown in Table 3 can be detected by RT-PCR using primers and probes, which are as described above. The probes are TaqMan probes. Preferably, the sequences of the primers and probes are shown in Table 6.

In an alternative embodiment, the RT-PCR method in step (2-3) is RT-PCR based on SYBR Green dye, and primers and commercially available SYBR Green premix can be used to detect the genes shown in Table 6, concurrently or separately. The primers are as described above. Preferably, the sequences of the primers are as shown in SEQ ID NO. 71 - SEQ ID NO. 88 (also see Table 6). The above RT-PCR detection can be performed with ABI 7500 real-time fluorescence quantitative PCR instrument (Applied Biosystems) or LightCycler® 480II from Roche. After the reaction, the Ct value of each gene is recorded, which represents the expression level of each gene.

In an embodiment, step (3) can be carried out by, for example, the following:
(3-1) Through survival analysis using a statistical software known in the art (such as x-tile software, SPSS or other analysis software that can be used to calculate the critical value, preferably x-tile software) based on the expression data of the genes in the gene panel according to the present disclosure in a statistically significant number of HER2-enriched or HER2-positive breast samples in combination with the survival data, the expression value that can distinguish the difference of the survival curves to the greatest extent is obtained as the critical value (cut-off value);
(3-2) Based on the critical value in step (3-1), the expression level of the gene obtained in step (2) in the subject sample is determined as strong (expression level > critical value) or weak (expression level ≤ critical value) value);
(3-3) The strength of the gene expression levels in step (3-2) is sufficient to provide assessment of the recurrence risk of breast cancer and/or guidance for breast cancer treatment with interferon in the subject.

After obtaining the data on the expression levels of the genes in the gene panel according to the present disclosure, those skilled in the art can apply technology known in the art and perform survival analysis in combination with survival data so as to obtain the critical value and judge the expression levels of the genes in in the gene panel according to the present disclosure as strong or weak.

In a preferable embodiment, step (3) can be carried out as follows:
(3-1) By analyzing the detection results of sequencing or quantitative PCR in step (2), in combination with survival data, and using a statistical software known in the art, the effect of each gene in the gene panel according to the present disclosure on the occurrence of breast cancer metastasis is obtained; according to the contribution of each gene to the effect on distant metastasis, a group of interferon pathway-related genes (i.e., the preferable interferon pathway-related genes) which contribute the most to the effect on distant metastasis are obtained, and an interferon index is calculated with the preferable interferon pathway-related genes using the weighting method;
(3-2) The interferon index is determined as strong or weak (for a detailed description of the method for calculating the interferon index and judging the strength of the interferon index, reference can be made to Examples 1 and 2);
(3-3) The strength of the interferon index in step (3-2) is sufficient to provide assessment of the recurrence risk of breast cancer and/or guidance for breast cancer treatment with interferon in the subject.

After obtaining the data on the expression levels of the genes in the gene panel according to the present disclosure, those skilled in the art can apply technology known in the art and perform survival analysis in combination with survival data so as to obtain the effect of each gene in the gene panel according to the present disclosure on occurrence of breast cancer metastasis, calculate the interferon index, and determine the interferon index as strong or weak.

The detection method according to the present disclosure can be used for a diagnostic purpose or non-diagnostic purpose.

Provided is also use of the gene panel or agent according to the present disclosure for the manufacture of a diagnostic product for assessing the recurrence risk of breast cancer and/or providing guidance for breast cancer treatment with interferon. The gene panel is described as above. The agent is described as above. Preferably the breast cancer is HER2-enriched or HER2-posistive breast cancer. In a preferable embodiment, the interferon is type I interferon.

In a preferable embodiment, the diagnostic product is in a form of a diagnostic kit.

Accordingly, in another aspect, provided is a diagnostic product for assessing the recurrence risk of breast cancer and/or providing guidance for breast cancer treatment with interferon, comprising the agent according to the present disclosure. Preferably the breast cancer is HER2-enriched or HER2-posistive breast cancer. In a preferable embodiment, the interferon is type I interferon.

The agent or diagnostic product according to the present disclosure can also be used in combination with an additional diagnostic product, including but not limited to breast cancer molecular subtyping diagnostic product and diagnostic product for detecting the expression level of HER2 in breast cancer. The breast cancer molecular subtyping diagnostic product can for example, be selected from PAM50 and the 72-gene panel for determining breast cancer molecular subtypes. The diagnostic product for detecting the expression level of HER2 in breast cancer can detect amplification and/or overexpression mRNA of the HER2 gene (e.g., based on a diagnostic product of quantitative PCR, DNA-FISH, RNA-FISH, CISH, Next-Generation Sequencing or gene chip) and/or HER2 protein overexpression (e.g., diagnostic product of IHC, ELISA or protein microarray).

The test sample used in the present disclosure is preferably a tissue from the test object (subject), as long as the total RNA of the subject can be extracted from the test sample. The test sample is preferably one or more selected from a tissue, blood, plasma and body fluid sample, more preferably a tissue sample, such as a paraffin tissue sample. In a preferable embodiment, the test sample is tissue with high content of tumor cells.

Exemplary embodiments are as follows.

1. A gene panel for assessing the recurrence risk of breast cancer and/or providing guidance for breast cancer treatment with interferon, comprising
at least one gene in Gene panel G1, and/or
at least one gene in Gene panel G2, and/or
at least one gene in Gene panel R; wherein
the Gene panel G1 comprises: IFI35, IFIT3, OAS2, OASL, RTP4 and SAMD9,
the Gene panel G2 comprises: OAS3, DDX58, SP110, IFIH1, DDX60 and XAF1,
the Gene panel R comprises: EIF2AK2, HERC5, HERC6, IFI27, IFI44, IFI44L, IFI6, IFIT1, IFIT5, IFITM1, ISG15, MX1, MX2, OAS1, PLSCR1, RSAD2 and USP18.

2. The gene panel according to embodiment 1, wherein the gene panel comprises SAMD9 and at least one of: IFI35, IFIT3, OAS2, OASL and RTP4.

3. The gene panel according to embodiment 1 or 2, wherein
the gene panel further comprises a reference gene(s),
preferably, the reference gene(s) comprises one, more preferably 3, most preferably 6 of: GAPDH, GUSB, MRPL19, PSMC4, SF3A1, TFRC, ACTB and RPLPO.

4. The gene panel according to any one of embodiments 1-3, wherein
the gene panel comprises: IFI35, IFIT3, OAS2, OASL, RTP4 and SAMD9, and optionally further comprises: ACTB, GAPDH and RPLPO; or
the gene panel comprises: OAS3, DDX58, SP110, IFIH1, DDX60 and XAF1, and optionally further comprises: ACTB, GAPDH and RPLPO; or
the gene panel comprises: DDX58, DDX60, EIF2AK2, HERC5, HERC6, IFI27, IFI35, IFI44, IFI44L, IFI6, IFIH1, IFIT1, IFIT3, IFIT5, IFITM1, ISG15, MX1, MX2, OAS1, OAS2, OAS3, OASL, PLSCR1, RSAD2, RTP4, SAMD9, SP110, USP18 and XAF1, and optionally further comprises: GAPDH, GUSB, MRPL19, PSMC4, SF3A1, TFRC.

5. The gene panel according to any one of embodiments 1-4, wherein

the breast cancer is HER2-enriched or HER2-posistive breast cancer.

6. The gene panel according to any one of embodiments 1-5, wherein

the interferon is type I interferon.

7. An agent for detecting the expression levels of the genes in the gene panel according to any one of embodiments 1-6.

8. The agent according to embodiment 7, being an agent for detecting the amount of RNA, particularly mRNA, transcribed from the gene; or an agent for detecting the amount of cDNA complementary to the mRNA.

9. The agent according to embodiment 7 or 8, being a primer(s), a probe(s) or a combination thereof.

10. The agent according to embodiment 9, wherein

the primer(s) and probe(s) have a sequence as shown in SEQ ID NO. 1 - SEQ ID NO. 97, and/or the primer(s) has a sequence as shown in SEQ ID NO. 1 - SEQ ID NO. 70.

11. The agent according to embodiment 10, wherein

the primer(s) has a sequence as shown in SEQ ID NO. 71 - SEQ ID NO. 88, and/or the probe(s) has a sequence as shown in SEQ ID NO. 89 -SEQ ID NO. 97.

12. The agent according to embodiment 11, wherein

the probe(s) is a TaqMan probe(s).

13. The agent according to embodiment 7, being an agent for detecting the amount of polypeptides encoded by the gene, preferably the agent is an antibody, an antibody fragment or an affinity protein.

14. A diagnostic product for assessing the recurrence risk of breast cancer and/or providing guidance for breast cancer treatment with interferon, comprising the agent according to any one of embodiments 7-13, preferably the breast cancer is HER2-enriched or HER2-posistive breast cancer.

15. The diagnostic product according to embodiment 14, wherein the diagnostic product further comprises a total RNA extraction reagent, a reverse transcription reagent, a Next-Generation Sequencing reagent and/or a quantitative PCR reagent.

16. The diagnostic product according to embodiment 14 or 15, wherein
the diagnostic product further comprises an additional diagnostic product,
preferably, the additional diagnostic product is a diagnostic product for determining the molecular subtype of breast cancer or determining the expression level of HER2 gene or protein in breast cancer, for example, the 72-gene panel for determining breast cancer molecular subtypes or PAM50.

17. The diagnostic product according to any one of embodiments 14-16, wherein

the diagnostic product is in a form of an in vitro diagnostic product, preferably in a form of a diagnostic kit.

18. The diagnostic product according to any one of embodiments 14-17, wherein

the diagnostic product is a Next-Generation Sequencing kit, a Real-time fluorescence quantitative PCR detection kit, a gene chip, a protein microarray, an ELISA diagnostic kit or an Immunohistochemistry (IHC) kit.

19. Use of the gene panel according to any one of embodiments 1-6 or the agent according to any one of embodiments 7-13 for the manufacture of a diagnostic product for assessing the recurrence risk of breast cancer and/or providing guidance for breast cancer treatment with interferon; preferably the breast cancer is HER2-enriched or HER2-posistive breast cancer.

### BENEFICIAL EFFECTS

Provided is a gene panel, an agent for detecting the expression levels of the genes in the gene panel, a method and a diagnostic product for assessing the recurrence risk of breast cancer and/or providing guidance for breast cancer treatment with interferon.

At present, multi-gene expression profiling products that can be used for assessing the recurrence risk of breast cancer and providing guidance for breast cancer clinical treatment include Oncotype DX, MammaPrint, PAM50, EndoPredict, the 72-gene panel for determining breast cancer molecular subtypes, or the like. Oncotype DX can be used to assess the recurrence risk in a patient with early-stage, estrogen receptor-positive breast cancer and to guide the clinical application of chemotherapy or endocrine therapy. MammaPrint can be used to assess risk of distant metastasis in a patient with lymph node-negative, estrogen receptor-negative or -positive early-stage breast cancer and guide the clinical application of chemotherapy. PAM50 categories breast cancer into four subtypes: Luminal A, Luminal B, Basal-like and HER2-enriched, and can guide chemotherapy or endocrine therapy in a patient with lymph node-negative, hormone receptor-positive and HER2-negative breast cancer. EndoPredict can be used to assess the risk of distant metastasis of ER-positive/HER2-negative breast cancer and guide the clinical application of postoperative chemotherapy. The 72-gene panel for determining breast cancer molecular subtypes categories breast cancer into Luminal A, Luminal B, Basal-like, HER2-enriched, and Immune-enhanced subtypes, and assesses 10-year recurrence risk of breast cancer according to tumor subtype, immune index and proliferation index.

The high heterogeneity of breast cancer leads to significant difference in the sensitivity and prognosis of different subtypes of breast cancers to endocrine therapy, targeted therapy or chemotherapy. For HER2-enriched or HER2-positive breast cancer, “anti-HER2-targeted therapy + chemotherapy” is the current gold standard for clinical treatment. However, due to the rapid progression and poor prognosis of HER2-related breast cancer, the treatment for HER2-enriched or HER2-positive breast cancer is difficult. On the other hand, the response of HER2-positive breast cancer to the treatment regimen of “anti-HER2-targeted therapy + chemotherapy” varies greatly. It has been shown in some studies that when PAM50 molecular subtyping is used for HER2-positive breast cancer, the HER2-enriched subtype accounts for the majority, but other molecular subtypes have a certain proportion. The anti-HER2-targeted therapy + chemotherapy treatment regimen shows the best therapeutic effect for the HER2-enriched subtype. Therefore, it is desirable to further subdivide breast cancer so as to improve the diagnosis and treatment efficiency of breast cancer. The diagnostic product provided herein can further subdivide HER2-enriched or HER2-positive breast cancer into two subgroups: strong interferon and weak interferon. For breast cancer with low interferon index and high recurrence risk, through combining targeted therapy and chemotherapy with interferon, it is expected to reduce the recurrence risk and improve the survival rate, not only improving the diagnosis and treatment efficiency of breast cancer, but also enhancing the efficiency of prediction of the recurrence risk of breast cancer.

The current diagnostic products mainly focus on HER2-negative breast cancer. The diagnostic product provided herein will provide benefit for breast cancer patients, especially those with HER2-enriched or HER2-positive breast cancer. In addition, in terms of clinical treatment guidance for breast cancer, although there are diagnostic products that predict whether patients with certain breast cancer subtype will benefit from chemotherapy or endocrine therapy regimen, the clinical treatment guidance for interferon therapy is not yet available. This issue will be solved by the present embodiments which can provide guidance for breast cancer clinical treatment application with interferon, especially for HER2-enriched or HER2-positive breast cancer. Another advantage of the present disclosure lies in that multiple selectable genes or gene combinations are provided as complementary embodiments. When the present embodiments are applied to cancer patients, if the expression level detection for one or some genes is invalid or fails due to pathological condition of the patients or other reasons (e.g., abnormal expression of one or some genes), multiple alternatives can be used as supplement, such that the detection results based on the present disclosure are more stable and reliable.

### EXAMPLES

The present disclosure is further described below by Examples, which do not limit the present disclosure to the scope of the Examples. The experimental procedures without specific conditions in the following Examples can be selected according to conventional methods and conditions or the manufacturer’s instructions.

### Example 1: Screening of a Gene Panel Affecting Distant Metastasis and Treatment Efficacy of HER2-Enriched Breast Cancer

### Experimental Procedures

By EPIG-supervised clustering analysis of gene expression profiles and clinical information in the breast cancer cohort study including 1655 cases in combination with the 72-gene panel for determining breast cancer molecular subtypes, a group of gene panels including interferon pathway-related genes were screened out. The expression levels of these genes were closely related to HER2-enriched breast cancer regarding the distant metastasis of breast cancer, but there is no significant correlation with the distant metastasis of breast cancer of other molecular subtypes, which may be used to provide guidance for breast cancer treatment with interferon of such subtype.

### Experimental Results

1. According to the results of Cox regression analysis, a total of 29 interferon pathway-related genes were obtained (see Table 4). To better illustrate the embodiments of the present disclosure, the 29 genes were grouped into G1, G2, R. However, embodiments of the present disclosure were not particularly limited to these grouped gene panels and gene panels used in the Examples.

TABLE 4

| Gene panel | Gene name | Cox regression coefficient | Hazard ratio | Cox regression p value |
|---|---|---|---|---|
| G1 | SAMD9 | -0.756 | 0.470 | 0.003 |
| | RTP4 | -0.568 | 0.567 | 0.002 |
| | OASL | -0.560 | 0.571 | 0.004 |
| | IFI35 | -0.492 | 0.611 | 0.008 |
| | IFIT3 | -0.465 | 0.628 | 0.007 |
| | OAS2 | -0.399 | 0.671 | 0.008 |
| G2 | OAS3 | -0.397 | 0.672 | 0.015 |
| | DDX58 | -0.307 | 0.736 | 0.035 |
| | SP110 | -0.306 | 0.737 | 0.185 |
| | IFIH1 | -0.276 | 0.759 | 0.067 |
| | DDX60 | -0.259 | 0.772 | 0.105 |
| | XAF1 | -0.257 | 0.774 | 0.118 |
| R | RSAD2 | -0.246 | 0.782 | 0.040 |
| | HERC5 | -0.221 | 0.801 | 0.206 |
| | MX2 | -0.221 | 0.801 | 0.255 |
| | IFI44 | -0.221 | 0.802 | 0.060 |
| | OAS1 | -0.206 | 0.814 | 0.176 |
| | IFIT5 | -0.201 | 0.818 | 0.344 |
| | IFI44L | -0.178 | 0.837 | 0.068 |
| | PLSCR1 | -0.169 | 0.845 | 0.301 |
| | IFI27 | -0.143 | 0.867 | 0.122 |
| | MX1 | -0.143 | 0.867 | 0.138 |
| | IFI6 | -0.128 | 0.880 | 0.189 |
| | HERC6 | -0.115 | 0.891 | 0.349 |
| | IFITM1 | -0.098 | 0.907 | 0.456 |
| | EIF2AK2 | -0.095 | 0.909 | 0.757 |
| | ISG15 | -0.086 | 0.918 | 0.362 |
| | IFIT1 | -0.076 | 0.927 | 0.361 |
| | USP18 | -0.048 | 0.954 | 0.824 |

2. The 29 interferon pathway-related genes in Table 4 were combined with 6 reference genes to form a set of 35-gene testing combination (see Table 2). From the 29 interferon pathway-related genes in Table 4, 6 genes most closely related to the distant metastasis of HER2-enriched breast cancer (preferably interferon pathway-related genes, see Table 4 for Gene panel G1) were selected and combined with 3 reference genes to form a set of 9-gene testing combination (see Table 3).

### Example 2: Assessing Recurrence Risk of Breast Cancer And/or Providing Guidance for Breast Cancer Treatment with Interferon Based on Interferon Index

Experimental procedures: The 1655 breast cancer tumor cases were subjected to molecular subtyping using the 72-gene panel for determining breast cancer molecular subtypes, and the expression levels of the interferon pathway-related genes were normalized based on the expression levels of reference genes (ACTB, GAPDH and RPLPO). The normalized expression levels of genes and their contribution to breast cancer metastasis were calculated using the weighted method to calculate the interferon index so as to assess effect of the interferon index on distant metastasis of breast cancer in different molecular subtypes.

2.1 Calculation of interferon index based on Gene panel G1, with the following formula: Unscaled interferon index (Unscaled Interferon Score, UIS)
UIS_{G1} = 0.23xSAMD9 + 0.17x(RTP4 + OASL)/2 + 0.15xIFI35 + 0.14xIFIT3 +0.12xOAS2
where the gene name represents the normalized expression level of the gene (for example, SAMD9 represents the normalized expression level of the SAMD9 gene), and the coefficient prior to the gene name represents the weighting factor of the gene (for example, 0.23 represents the weighting factor of the SAMD9 gene).

Interferon index (Interferon Score, IS)
Interferon index (IS)= 30xUIS + 26
Weak interferon index: IS 1-32; strong interferon index: IS 33-100.

### Experimental Results

### 1. Effect of Interferon Index on Recurrence Risk of HER2-Enriched Breast Cancer

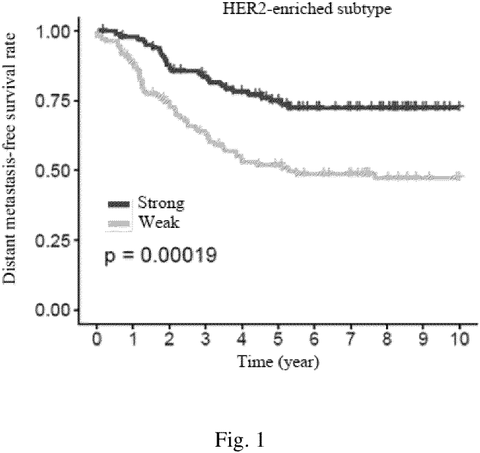

According to the calculated interferon index, the breast cancer cases were categorized into two groups as strong interferon index and weak interferon index. In HER2-enriched breast cancer cases, the 10-year distant metastasis-free survival rate of the group with strong interferon index was significantly higher than that of the group with weak index (P<0.001) (FIG. 1), indicating that HER2-enriched breast cancer patients with strong interferon index have a lower recurrence risk and better prognosis. For HER2-enriched breast cancer patients with weak interferon index, it is possible to reduce the recurrence risk of breast cancer through combination therapy with interferon to enhance the interferon signaling pathway.

### 2. Effect of Interferon Index on Recurrence Risk on Other Molecular Subtypes of Breast Cancer

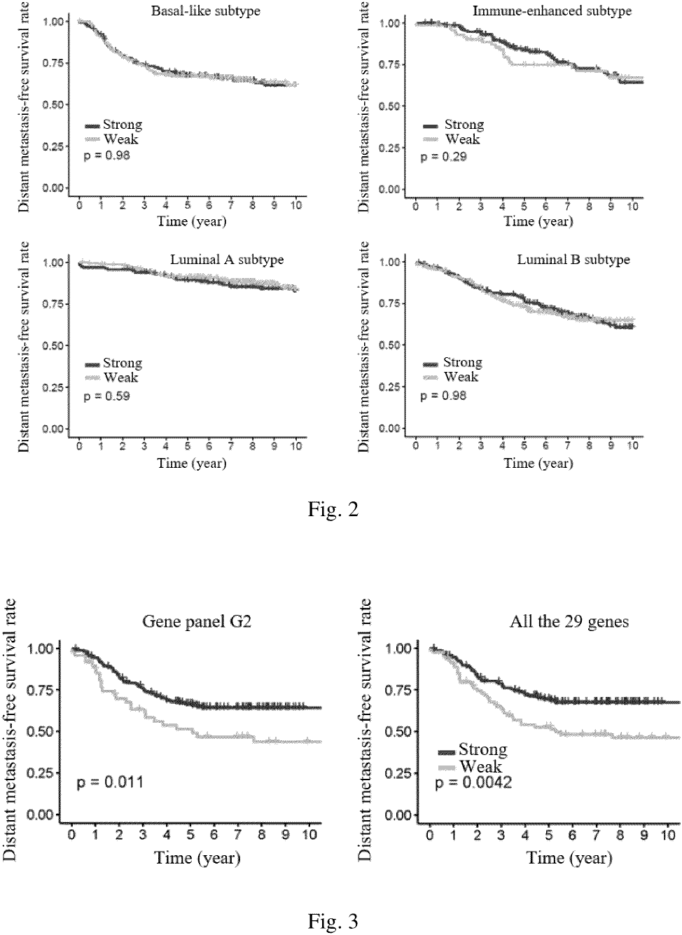

Also grouped by the interferon index, for Luminal A, Luminal B, Basal-like and Immune-enhanced breast cancer, there was no significant difference in 10-year distant metastasis-free survival rate between the group of strong interferon index and the group of weak interferon index (FIG. 2), indicating that there is no significant correlation between the strength of interferon index and the recurrence risk for breast cancer patients of such subtypes, and the strength of interferon index does not affect prognosis of breast cancer patients of such subtypes.

### 2.2 Calculation of Interferon Index Based on the 6 Genes in Gene Panel G2 or All the 29 Genes as Shown in Table 4, with the Following Formula

Unscaled interferon index (Unscaled Interferon Score, UIS)

Gene panel G2:

$$\begin{matrix}{\text{UIS}}_{\text{G} 2} = 0.22 \text{xOAS3 + 0} \text{.17x} \left(\text{DDX} 58 + \text{SP} 110\right) / \text{} \\ 2 + 0.15 \text{xIFIH1} + 0.14 \text{x} \left(\text{DDX60}\right) + \left(\text{XAF1}\right) / 2\end{matrix}$$

Based on all the 29 genes:

$$\begin{matrix}{\text{UIS}}_{29} = 0.098 \text{xSAMD9+0} \text{.074x} \left(\text{RTP4+OASL}\right) / 2 + \\ 0.062 \text{x} \left(\text{IFI35+IFIT3}\right) / 2 + 0.052 \text{x} \left(\text{OAS2}\right) + \left(\text{OAS3}\right) / 2 + \\ 0.040 \text{x} \left(\text{DDX58} + \text{SP110}\right) / 2 + \\ 0.034 \text{x} \left(\text{IFIH1 + DDX60 + XAF1 + RSAD2}\right) / 4 + \\ 0.028 \text{x} \left(\text{HERC5 + MX2 + IFI44 + OAS1 +IFIT5}\right) / 5 + \\ 0.023 \text{x} \left(\text{IFI44L + PLSCR1}\right) / 2 + \\ 0.017 \text{x} \left(\text{IFI27 + MX1 + IFI6 + HERC6}\right) / 4 + \\ 0.012 \text{x} \left(\text{IFITM1 + EIF2AK2 + ISG15 + IFIT1}\right) / 4 + \\ 0.006 \text{xUSP18}\end{matrix}$$

Interferon index (Interferon Score, IS)
Interferon index (IS)= 30xUIS + 26
Weak interferon index: IS 1-32; Strong interferon index: IS 33-100.

### 1. Effect of Interferon Index on Recurrence Risk of HER2-Enriched Breast Cancer

According to the calculated interferon index, breast cancer cases were categorized into two groups as strong interferon index and weak interferon index. In HER2-enriched breast cancer cases, for the group with strong interferon index based on Gene panel G2 or the group with strong interferon index based on all the 29 genes, the 10-year distant metastasis-free survival rate was higher than that of the group with weak index (FIG. 3), indicating that HER2-enriched breast cancer patients with strong interferon index have a lower recurrence risk and better prognosis. For HER2-enriched breast cancer patients with weak interferon index, it is possible to reduce the recurrence risk of breast cancer through combination therapy with interferon to enhance the interferon signaling pathway.

### 2. Effect of Interferon Index on Recurrence Risk of Other Molecular Subtypes of Breast Cancer

With respect to all interferon index for Luminal A, Luminal B, Basal-like and Immune-enhanced breast cancer, there was no significant difference in 10-year distant metastasis-free survival rate between the group of strong interferon index and the group of weak interferon index.

### Example 3: Assessing Recurrence Risk And/or Providing Guidance for Breast Cancer Treatment with Interferon for HER2-Enriched Breast Cancer Based on Their Respective Expression Levels of Interferon Pathway Genes

### Experimental Procedures

The 1655 breast cancer cases were subjected to molecular subtyping using the 72-gene panel for determining breast cancer molecular subtypes, and the expression levels of interferon pathway-related genes were normalized based on the expression levels of reference genes (ACTB, GAPDH and RPLPO) so as to assess effect of the expression level of each gene in Gene panel G1 according to the present disclosure (see Table 4) on distant metastasis of HER2-enriched breast cancer, with the following steps:
(i) Through survival analysis using the x-tile software based on the distribution of expression level of each gene in a population of HER2-enriched breast cancer patients in combination with the survival data, the expression value that could distinguish the difference in the survival curves to the greatest extent was obtained as the critical value;
(ii) Based on the critical value, the expression level of the gene was determined as strong or weak;
(iii) Assessment of the recurrence risk of breast cancer and/or providing guidance for breast cancer treatment with interferon in the subject were performed according to the strength of the gene expression level.

### 3.1 Assessment of the Recurrence Risk and/or Providing Guidance for Breast Cancer Treatment with Interferon Were Performed for HER2-Enriched Breast Cancer According to the Expression Level of Each of the 6 Genes in Gene Panel G1 (See Table 4)

### Experimental Results

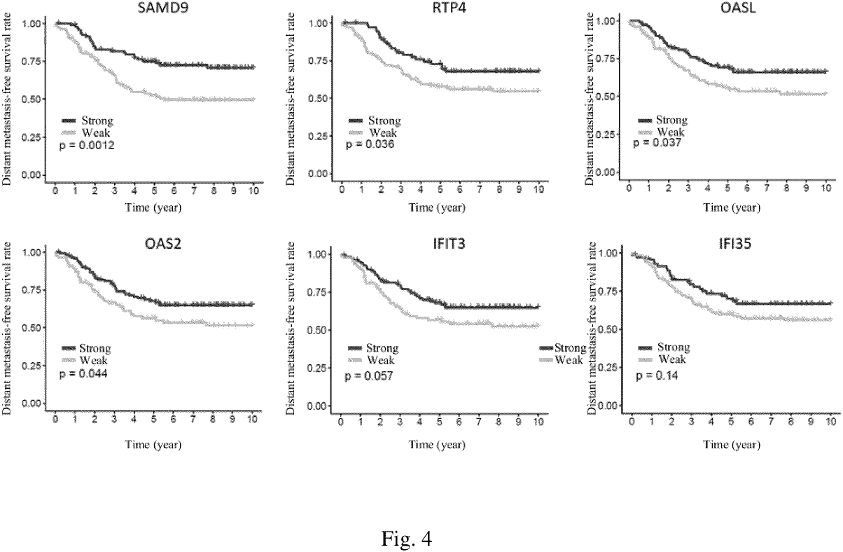

According to expression level score of each of the 6 genes (see Table 4) in Gene panel G1, HER2-enriched breast cancer were categorized into two groups as strong expression level and weak expression level, where the 10-year distant metastasis-free survival rate in the group with strong expression level of each gene was higher than that of the group with weak gene expression level (FIG. 4), indicating that HER2-enriched breast cancer patients with strong expression levels of the preferable interferon pathway-related genes have a lower recurrence risk and better prognosis. For HER2-enriched breast cancer patients with weak expression levels of the interferon pathway-related genes, it is possible to reduce the recurrence risk of breast cancer through combination therapy with interferon to enhance the interferon signaling pathway.

### 3.2 Assessing the Recurrence Risk And/or Providing Guidance for Breast Cancer Treatment with Interferon for HER2-Enriched Breast Cancer According to the Expression Level of Each Gene in GENE Panel G2 or Gene Panel R (See Table 4)

### Experimental Results

Effect of the expression levels of the genes in Gene panel G2 or Gene panel R on the 10-year distant metastasis-free survival rate of HER2-enriched breast cancer was similar as that of the genes in Gene panel G1, but the influence efficiency was relatively less than that of the genes in Gene panel G1 (Table 4).

### Example 4: Assessing the Recurrence Risk And/or Providing Guidance for Breast Cancer Treatment with Interferon for HER2-Positive Breast Cancer Based on the Strength of Interferon Index

Experimental procedures: the 1655 breast cancer cases were categorized into HER2-positive and HER2-negative groups to assess effect of interferon index on occurrence of distant metastasis of breast cancer.

The interferon index was calculated based on Gene panel G1, where the calculation method was as that of Example 2.1.

### Experimental Results

### 1. Effect of Interferon Index on Recurrence Risk of HER2-Positive Breast Cancer

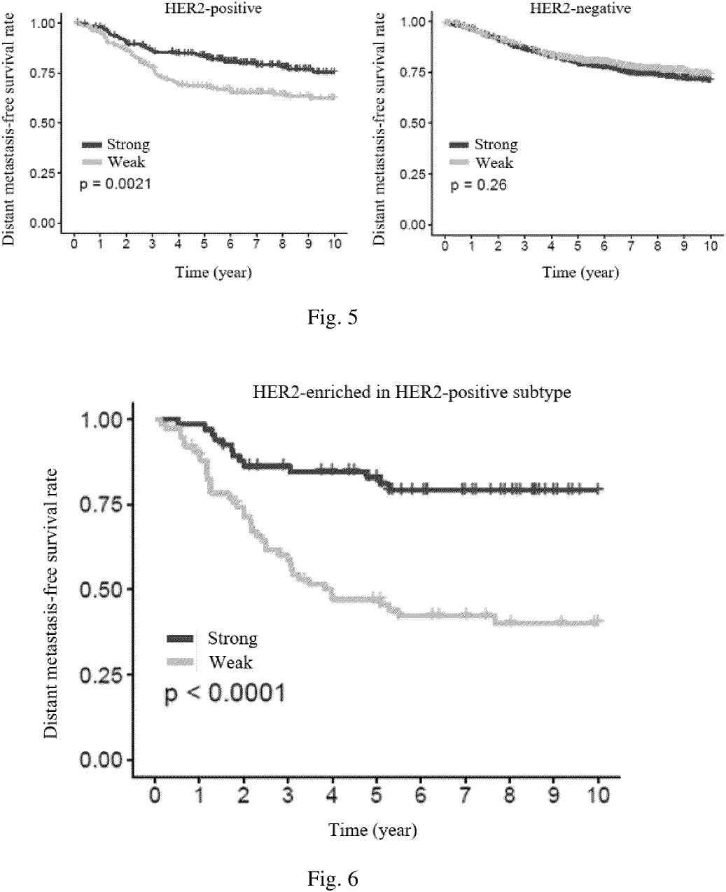

In HER2-positive breast cancer cases, 10-year distant metastasis-free survival rate of the group with strong interferon index was significantly higher than that of the group with weak index (P<0.001) (FIG. 5, left), indicating that the HER2-positive breast cancer patients with strong interferon index have a lower recurrence risk and better prognosis. For HER2-positive breast cancer patients with weak interferon index, it is possible to reduce the recurrence risk of breast cancer through combination therapy with interferon to enhance the interferon signaling pathway.

### 2. Effect of Interferon Index on Recurrence Risk of HER2-Negative Breast Cancer

In the HER2-negative breast cancer cases, there was no significant difference in 10-year distant metastasis-free survival rate between the case groups with strong interferon index and weak interferon index(FIG. 5, right), indicating that the strength of interferon index is not significantly related with the recurrence risk of HER2-negative breast cancer patients and does not affect the prognosis of patients with HER2-negative breast cancer.

### Example 5: Assessing the Recurrence Risk And/or Providing Guidance for Breast Cancer Treatment with Interferon for HER2-Enriched Breast Cancer in HER2-Positive Breast Cancer Based on the Strength of Interferon Index

Experimental procedures: HER2-positive breast cancers of the 1655 breast cancer cases in Example 4 were subjected to molecular subtyping using the 72-gene panel for determining breast cancer molecular subtypes to assess effect of interferon index on occurrence of distant metastasis of breast cancer.

The interferon index was calculated based on Gene panel G1, where the calculation method was as that of Example 2.1.

### Experimental Results

According to the calculated interferon index, the cases classified as HER2-enriched breast cancer cases can be categorized into two groups as strong interferon index and weak interferon index, where the 10-year distant metastasis-free survival rate of the group with strong interferon index was significantly higher than that of the group with weak index (P<0.001) (FIG. 6), indicating that HER2-positive breast cancer patients with strong interferon index have a lower recurrence risk and better prognosis. In addition, when the 72-gene panel for determining breast cancer molecular subtypes was used for further molecular subtyping on HER2-positive breast cancer followed by recurrence risk assessment, compared with HER2-enriched (FIG. 1) or HER2-positive (FIG. 5 left) breast cancer alone, the prognosis prediction efficiency was significantly improved. For HER2-enriched breast cancer patients in HER2-positive breast cancer with weak interferon index, it is possible to reduce the recurrence risk of breast cancer through combination therapy with interferon to enhance the interferon signaling pathway.

### Example 6: Analysis of Interferon Pathway-Related Gene Panel Via Next-Generation Sequencing Detection Kit

Experimental procedures: Breast cancer tumor tissue was taken to extract RNA from the tumor cells. Illumina Next-Generation Sequencing (NGS) technology was used to design and optimize 35 pairs of primers (see Table 5) corresponding to the 35 genes shown in Table 2 and the expression levels were detected, respectively, with the following steps:
(1): Tumor or paraffin-embedded tissue of the subject was taken to obtain the area with high tumor cells as the original material.
(2): Total RNA was extracted from the tissue, for example, using RNA extraction kit of Cell Data Sciences (RNA storm CD201) or RNA extraction kit of Qiagen (Qiagen RNease FFPE kit, Cat. No. 73504).
(3): The obtained RNA was prepared into a library used for Next-Generation Sequencing of targeted RNA-seq technology. The preparation method of the library comprises the following steps:
   (3-1): The RNA extracted in step (2) was reverse transcribed into cDNA using ProtoScript® II Reverse Transcriptase (New England Biolabs, #M0368L).
   (3-2): TruSeq® Targeted RNA Library Construction Kit (#15034457) from Illumina was used to prepare the obtained cDNA into a library for sequencing, with the following specific steps: (i) Hybridization: adding 4.5 µl of TOP (see Table 5 for the specific compositions), adding 21 µl of OB1 after mixing, heating to 70° C. and then slowly cooling down to 30° C.; (ii) extension and ligation: adsorbing the product in (i) on a magnetic stand and discarding the supernatant, washing twice with AM1 and UB1 in the kit, discarding the supernatant, adding 36 µl of ELM4, and incubating at 37° C. for 45 min in in a PCR instrument or metal bath; (iii) subjecting the product obtained in (ii) to ligation with a sequencing tag (Index), followed by PCR: absorbing the product obtained in (ii) on a magnetic stand, discarding the supernatant, adding 18 µl of 40-fold diluted HP3, 16 µl of which was taken after absorption with a magnetic stand and added with 17.3 µl of TDP1, 0.3 µl of PMM2, 6.4 µl of Index and mixing well, followed by PCR amplification for 32 cycles; (iv) purifying the DNA with Gnome DNA purification kit (QuestGenomics, Nanjing) to obtain the library.
(4): The obtained DNA library was subjected to Next-Generation Sequencing with NextSeq/MiSeq/MiniSeq/iSeq. Paired-end or single-end sequencing was performed with Illumina NextSeq/MiSeq/MiniSeq/iSeq sequencers.
(5): Statistical analysis of the results. Based on the expression levels of the reference genes, the obtained detection results were normalized to calculate the gene expression levels. The method for judging the gene expression levels was as shown in Example 3.

TABLE 5

| No. | Gene name | Gene ID | Sequence of primers in upstream region of gene | Sequence of primers in downstream region of gene |
|---|---|---|---|---|
| 1 | DDX58 | NM_014314 | GAGCTACATGGCCCCCTGG TTTAG (SEQ ID NO. 1) | TTCAGGCTGAGAAAAACA ACAAGGGC (SEQ ID NO. 2) |
| 2 | DDX60 | NM_017631 | GAAGGATTTTGCACTCACC ATTAA (SEQ ID NO. 3) | TGCGTGAGCTATGTGAAAA TGAAGAC (SEQ ID NO. 4) |
| 3 | EIF2AK2 | NM_001135 651 | CAAATTAGCTGTTGAGATA CTTAA (SEQ ID NO. 5) | CTTTATTATTGACAACAAC GAATTCT (SEQ ID NO. 6) |
| 4 | HERC5 | NM_016323 | GAGATTACCATTCTCTTGC ACTCT (SEQ ID NO. 7) | GGTGAGCTTTTTGCCTGGG GACAGAA (SEQ ID NO. 8) |
| 5 | HERC6 | NM_001165 136 | CACTACCACTCCCTGGCAT TATCA (SEQ ID NO. 9) | GCCAAGTGTTTTCGTGGGG AAAGAAC (SEQ ID NO. 10) |
| 6 | IFI27 | NM_001130 080 | TCAGCTTCACATTCTCAGG AACTC (SEQ ID NO. 11) | TCTGGCTGAAGTTGAGGAT CTCTTAC (SEQ ID NO. 12) |
| 7 | IFI35 | NM_005533 | GAGACCCATGTCAGCCCCA CTGGA (SEQ ID NO. 13) | GCCGCCCTCCACGCCCTTC AGGAGGA (SEQ ID NO. 14) |
| 8 | IFI44 | NM_006417 | ACTTCTTAGAGGATTTGCC TTTTG (SEQ ID NO. 15) | GAAATTATCAACTGTGCAC AAGGAAA (SEQ ID NO. 16) |
| 9 | IFI44L | NM_006820 | TTTTTAGAAGATTTGCCTCT TGAG (SEQ ID NO. 17) | TGGTGCAATTGAGAGAGC GTTACAGC (SEQ ID NO. 18) |
| 10 | IFI6 | NM_002038 | CTAGTGGCCACGCTGCAGA GCCTC (SEQ ID NO. 19) | GCAGCAGCGTCGTCATAGG TAATATT (SEQ ID NO. 20) |
| 11 | IFIH1 | NM_022168 | GAGGAACTGTTGACAATTG AAGAC (SEQ ID NO. 21) | GAAAACAATGGAAATGAAT CAGGTGT (SEQ ID NO. 22) |
| 12 | IFIT1 | NM_001270 927 | CAGAACGGCTGCCTAATTT ACAGC (SEQ ID NO. 23) | ATCAGGTCAAGGATAGTCT GGAGCAA (SEQ ID NO. 24) |
| 13 | IFIT3 | NM_001031 683 | CAGCAGAGACACAGAGGG CAGTCA (SEQ ID NO. 25) | GGTCACCAAGAATTCCCTG GAGAAAA (SEQ ID NO. 26) |
| 14 | IFIT5 | NM_012420 | TGATGGACATGTGAAAAGT AAGTA (SEQ ID NO. 27) | ATTAGGCTAATAGGCTTAA CCCAGGA (SEQ ID NO. 28) |
| 15 | IFITM1 | NM_003641 | GGTCTCACTGAGCACCGTC CCAGC (SEQ ID NO. 29) | AGCGGCCCTTCGCTCCACG CAGAAAA (SEQ ID NO. 30) |
| 16 | ISG15 | NM_005101 | GCCGTGGCCCACAGCCCA CAGCCC (SEQ ID NO. 31) | GGCTGGGACCTGACGGTG AAGATGCT (SEQ ID NO. 32) |
| 17 | MX1 | NM_001144 925 | TGCTACTGTGGCCCAGAAA AATCC (SEQ ID NO. 33) | CTGAGAACAACCTGTGCA GCCAGTAT (SEQ ID NO. 34) |
| 18 | MX2 | NM_002463 | GAAATAGGCATCCACCTGA ATGCC (SEQ ID NO. 35) | GTCTCGCCAACCAGATCCC ATTTATA (SEQ ID NO. 36) |
| 19 | OAS1 | NM_001032 409 | GCTCCTACCCTGTGTGTGT GTCCA (SEQ ID NO. 37) | GGGTGGCTCCTCAGGCAA GGGCACCA (SEQ ID NO. 38) |
| 20 | OAS2 | NM_001032 731 | CCGAACAGTTCCCCCTGGT GCAGG (SEQ ID NO. 39) | GCTCCTATGGACGGAAAAC AGTCTTA (SEQ ID NO. 40) |
| 21 | OAS3 | NM_006187 | CCCGCGGGTGCTGAAAAC TGTCAA (SEQ ID NO. 41) | GGCACAGCTCTCAAGGGT GGCTGTGA (SEQ ID NO. 42) |
| 22 | OASL | NM_001261 825 | TGTGCGGGTGCTGAAGGTA GTCAA (SEQ ID NO. 43) | CGGGAATGGCACGGTTCTC AGGAGCA (SEQ ID NO. 44) |
| 23 | PLSCR1 | NM_021105 | TCCTCAGTATCCACCGACA GCATT (SEQ ID NO. 45) | CTCCAGGATATAGTGGCTA CCCTGGG (SEQ ID NO. 46) |
| 24 | RSAD2 | NM_080657 | AGCAAAGAGAGGATTGCT TTTGCT (SEQ ID NO. 47) | GGAGAAGATCAACTTTTCA GGTGGAG (SEQ ID NO. 48) |
| 25 | RTP4 | NM_022147 | ATTTCTGGACTTGGGAGCA GACAT (SEQ ID NO. 49) | GAACTAATCCAAGAGGCA AAACCCCG (SEQ ID NO. 50) |
| 26 | SAMD9 | NM_001193 307 | TTTCCCCACATATATGTAAA AGAA (SEQ ID NO. 51) | CTCATGGTTGCAGTGGGTG AATAGGT (SEQ ID NO. 52) |
| 27 | SP110 | NM_001185 015 | GACAACTCCATCATCACTA AGAGA (SEQ ID NO. 53) | CTGGAAGCCTGTAGAAATT TGATCCC (SEQ ID NO. 54) |
| 28 | USP18 | NM_017414 | CCCAGGGCCTGGGACTACC CTCAT (SEQ ID NO. 55) | CAACATTGGACAGACCTGC TGCCTTA (SEQ ID NO. 56) |
| 29 | XAF1 | NM_017523 | AACATGGAAGGAGACTTCT CGGTG (SEQ ID NO. 57) | TGCCAACTTCACCCTCCAT GAGGCTT (SEQ ID NO. 58) |
| 30 | GAPDH | NM_002046 | TGAGGTCCACCACCCTGTT GCTGTA (SEQ ID NO. 59) | ATGAGCTTGACAAAGTGGT CGTTGA (SEQ ID NO. 60) |
| 31 | GUSB | NM_000181 | ATTGAAGCTGGAGGGAAC TGGCATG (SEQ ID NO. 61) | GCGGCCGCCGGTACCACTG CTCCTC (SEQ ID NO. 62) |
| 32 | MRPL19 | NM_014763 | CTGTTCTTCCCCTTCGAGG AATGAA (SEQ ID NO. 63) | TCCACGGGGCGGTGCTTGT CCACGA (SEQ ID NO. 64) |
| 33 | PSMC4 | NM_006503 | TCTGGGGCCGGGACACGG ACAGTGC (SEQ ID NO. 65) | CTTCTCCACCAAGATGCCT ATCTCC (SEQ ID NO. 66) |
| 34 | SF3A1 | NM_005877 | GAATCCTCCTTTGAAGATG CTTCTT (SEQ ID NO. 67) | GGCTGTTTGGGCTCCGTGG GCACGG (SEQ ID NO. 68) |
| 35 | TFRC | NM_003234 | CTTTTGGAGATACGTAGGG AGAGAG (SEQ ID NO. 69) | CACGATCATTGAGTTTCTT CATGAC (SEQ ID NO. 70) |

### Example 7: Analysis of Interferon Pathway-Related Gene Panel via RT-PCR Detection Kit

Experimental procedures: Breast cancer tumor tissue was taken to extract RNA from the tumor cells. TaqMan RT-PCR technology was used to design and optimize 9 pairs of primers and 9 TaqMan probes (see Table 6) corresponding to the 9 genes shown in Table 3 and the expression levels were detected, respectively, with the following steps:
(1): Fresh or paraffin-embedded tumor tissue of the subject was taken to obtain the area with high tumor cells as the original material.
(2): Total RNA was extracted from the tissue using RNA storm CD201RNA or Qiagen RNease FFPE kit RNA extraction kit.
(3): RT-PCR detection. The RT-PCR detection method was TaqMan RT-PCR, and the genes shown in Table 3 (see also Table 6) were subjected to RT-PCR detection, respectively, with the following steps:
   (3-1): Total RNA of the subject was extracted;
   (3-2): The RNA obtained in (3-1) was subjected to reverse transcription with the specific steps of: taking a total amount of about 2 µg of sample RNA (for example, taking 11 µl of sample RNA of about 200 ng/µl), and subjecting it to reverse transcription together with 11 µl of reference RNA (Thermo K1622 Reverse Transcription Kit) to obtain the sample cDNA and reference cDNA; adding 80 µl of RNase-free water to the sample cDNA for 5-fold dilution, and adding 180 µl of RNase-free water to the reference cDNA for 10-fold dilution;
   (3-3): The cDNA samples corresponding to each gene obtained in (3-2) was subjected to TaqMan RT-PCR to detect the 6 target genes and the reference genes (see Table 6) respectively, with the following steps: (i) Preparation of reaction system in each well: 2 µl of cDNA sample (total amount of 100-400 ng) obtained in (3-2), a total of 1.4 µl of forward and reverse specific primers and TaqMan fluorescent probe (10 µM) as shown in Table 6, 10 µl of reaction premix solution, and 6.6 µl of DEPC water; (ii) The reverse transcriptase was inactivated at 95° C. for 2 min; (iii) Amplification and detection: denaturation at 95° C. for 25 s, annealing, extension and fluorescence detection at 60° C. for 60 s for 45 cycles, and a holding period at 60° C. for 60 s; After the amplification reaction was completed, the Ct value of each gene was recorded, representing the expression level of each gene.
(4): Statistical analysis of the results. Based on the expression levels of the reference genes, the obtained detection results were normalized to calculate the interferon index. The method for calculating the interferon index and the method for judging the strength of the interferon index were as shown in Example 2.

TABLE 6

| No | Gene name | Gene ID | Forward primer sequence | Reverse primer sequence | Probe sequence |
|---|---|---|---|---|---|
| 1 | SAMD 9 | NM_001 193307 | GGGCACCTACTCTA CAATTCTG (SEQ ID NO. 71) | CCAGTGAGAACTC TGCAATCA (SEQ ID NO. 72) | AATGTGGGAACTA CTGTGGAGTACGC (SEQ ID NO. 89) |
| 2 | RTP4 | NM_022 147 | CCCAATATGAGATG CCTGAGTT (SEQ ID NO. 73) | GAGACTTCCTCGT GCCATTT (SEQ ID NO. 74) | TCTGAGCAACCTG GTGCAGCATAT (SEQ ID NO. 90) |
| 3 | OASL | NM_001 261825 | GCCATGTACTCCAG AACTCATC (SEQ ID NO. 75) | GGCCTGGGATAAC TCATTGTAA (SEQ ID NO. 76) | AACTGGTGAAGAC CTGGGACCTTC (SEQ ID NO. 91) |
| 4 | OAS2 | NM_001 032731 | CATAGACCCTCAG GAGAGAAGA (SEQ ID NO. 77) | TCCAAAGACAATC AGGGTATGG (SEQ ID NO. 78) | TGGACTCGCTCTCT TCTCTGGAACT (SEQ ID NO. 92) |
| 5 | IFIT3 | NM_001 031683 | AGTGGCTCATGCCT GTAATC (SEQ ID NO. 79) | AGACGGGATTTCA CTGTGTTAG (SEQ ID NO. 80) | CAAACTCCAGACC TCGTGATCCGC (SEQ ID NO. 93) |
| 6 | IFI35 | NM_005 533 | GAGCCTCCTGAGG TGTATTTC (SEQ ID NO. 81) | CACAGCAACTGAA CTCTTGTTT (SEQ ID NO. 82) | CAAAGGGCTGTGG TCTCTCTCAGC (SEQ ID NO. 94) |
| 7 | ACTB | NM_001 101 | GGATCAGCAAGCA GGAGTATG (SEQ ID NO. 83) | AGAAAGGGTGTAA CGCAACTAA (SEQ ID NO. 84) | TCGTCCACCGCAA ATGCTTCTAGG (SEQ ID NO. 95) |
| 8 | GAPD H | NM_002 046 | GGTGTGAACCATG AGAAGTATGA (SEQ ID NO. 85) | GAGTCCTTCCACG ATACCAAAG (SEQ ID NO. 86) | GGTGTGAACCATG AGAAGTATGA (SEQ ID NO. 96) |
| 9 | RPLP0 | NM_001 002 | GGAGAAACTGCTG CCTCATATC (SEQ ID NO. 87) | CAGCAGCTGGCAC CTTATT (SEQ ID NO. 88) | TGGTGAACACAAA GCCCACATTCC (SEQ ID NO. 97) |

1. A gene panel for assessing the recurrence risk of breast cancer and/or providing guidance for breast cancer treatment with interferon, comprising
at least one gene in Gene panel G1, and/or
at least one gene in Gene panel G2, and/or
at least one gene in Gene panel R; wherein
the Gene panel G1 comprises: SAMD9, IFI35, IFIT3, OAS2, OASL and RTP4,
the Gene panel G2 comprises: OAS3, DDX58, SP110, IFIH1, DDX60 and XAF1,
the Gene panel R comprises: EIF2AK2, HERC5, HERC6, IFI27, IFI44, IFI44L, IFI6, IFIT1, IFIT5, IFITM1, ISG15, MX1, MX2, OAS1, PLSCR1, RSAD2 and USP18.

2. The gene panel according to claim 1, wherein the gene panel comprises SAMD9 and at least one of: IFI35, IFIT3, OAS2, OASL and RTP4.

3. The gene panel according to claim 1 or 2, wherein
the gene panel further comprises a reference gene(s),
preferably, the reference gene(s) comprises one, more preferably 3, most preferably 6 of: GAPDH, GUSB, MRPL19, PSMC4, SF3A1, TFRC, ACTB and RPLP0.

4. The gene panel according to any one of claims 1-3, wherein
the gene panel comprises: SAMD9, IFI35, IFIT3, OAS2, OASL and RTP4, and optionally further comprises ACTB, GAPDH and RPLP0; or
the gene panel comprises: OAS3, DDX58, SP110, IFIH1, DDX60 and XAF1, and optionally further comprises ACTB, GAPDH and RPLP0; or
the gene panel comprises: SAMD9, IFI35, IFIT3, OAS2, OASL, RTP4, OAS3, DDX58, SP110, IFIH1, DDX60, XAF1, EIF2AK2, HERC5, HERC6, IFI27, IFI44, IFI44L, IFI6, IFIT1, IFIT5, IFITM1, ISG15, MX1, MX2, OAS1, PLSCR1, RSAD2 and USP18, and optionally further comprises GAPDH, GUSB, MRPL19, PSMC4, SF3A1, TFRC.

5. The gene panel according to any one of claims 1-4, wherein
the breast cancer is HER2-enriched or HER2-posistive breast cancer.

6. The gene panel according to any one of claims 1-5, wherein
the interferon is type I interferon.

7. An agent for detecting the expression levels of the genes in the gene panel according to any one of claims 1-6.

8. The agent according to claim 7, being
an agent for detecting the amount of RNA, particularly mRNA, transcribed from the genes; or
an agent for detecting the amount of cDNA complementary to the mRNA.

9. The agent according to claim 7 or 8, being a primer(s), a probe(s) or a combination thereof.

10. The agent according to claim 9, wherein
the primer(s) has a sequence as shown in SEQ ID NO. 1 - SEQ ID NO. 58, SEQ ID NO. 1 - SEQ ID NO. 70, SEQ ID NO. 71 - SEQ ID NO. 82 or SEQ ID NO. 71 - SEQ ID NO. 88.

11. The agent according to claim 9 or 10, wherein
the probe(s) has a sequence as shown in SEQ ID NO. 89 -SEQ ID NO. 94 or SEQ ID NO. 89 -SEQ ID NO. 97.

12. The agent according to any one of claims 9-11, wherein
the probe(s) is a TaqMan probe(s).

13. The agent according to claim 7, being an agent for detecting the amount of polypeptides encoded by the genes, preferably the agent is an antibody, an antibody fragment or an affinity protein.

14. A diagnostic product for assessing the recurrence risk of breast cancer and/or providing guidance for breast cancer treatment with interferon, comprising the agent according to any one of claims 7-13, preferably the breast cancer is HER2-enriched or HER2-posistive breast cancer.

15. The diagnostic product according to claim 14, wherein the diagnostic product further comprises a total RNA extraction reagent, a reverse transcription reagent, a Next-Generation Sequencing reagent and/or a quantitative PCR reagent.

16. The diagnostic product according to claim 14 or 15, wherein
the diagnostic product further comprises an additional diagnostic product,
preferably, the additional diagnostic product is a diagnostic product for determining the molecular subtype of breast cancer or determining the expression level of HER2 gene or HER2 protein in breast cancer, for example, the 72-gene panel for determining breast cancer molecular subtypes or PAM50.

17. The diagnostic product according to any one of claims 14-16, wherein
the diagnostic product is in a form of an in vitro diagnostic product, preferably in a form of a diagnostic kit.

18. The diagnostic product according to any one of claims 14-17, wherein
the diagnostic product is a Next-Generation Sequencing kit, a Real-time fluorescence quantitative PCR detection kit, a gene chip, a protein microarray, an ELISA diagnostic kit or an Immunohistochemistry (IHC) kit.

19. Use of the gene panel according to any one of claims 1-6 or the agent according to any one of claims 7-13 for the manufacture of a diagnostic product for assessing the recurrence risk of breast cancer and/or providing guidance for breast cancer treatment with interferon; preferably the breast cancer is HER2-enriched or HER2-posistive breast cancer.


SEQUENCE LISTING (97 sequences)

SEQ ID NO: 1 - DNA, 24 bp, Artificial Sequence
  DDX58 upstream primer sequence
gagctacatggccccctggtttag

SEQ ID NO: 2 - DNA, 26 bp, Artificial Sequence
  DDX58 downstream primer sequence
ttcaggctgagaaaaacaacaagggc

SEQ ID NO: 3 - DNA, 24 bp, Artificial Sequence
  DDX60 upstream primer sequence
gaaggattttgcactcaccattaa

SEQ ID NO: 4 - DNA, 26 bp, Artificial Sequence
  DDX60 downstream primer sequence
tgcgtgagctatgtgaaaatgaagac

SEQ ID NO: 5 - DNA, 24 bp, Artificial Sequence
  EIF2AK2 upstream primer sequence
caaattagctgttgagatacttaa

SEQ ID NO: 6 - DNA, 26 bp, Artificial Sequence
  EIF2AK2 downstream primer sequence
ctttattattgacaacaacgaattct

SEQ ID NO: 7 - DNA, 24 bp, Artificial Sequence
  HERC5 upstream primer sequence
gagattaccattctcttgcactct

SEQ ID NO: 8 - DNA, 26 bp, Artificial Sequence
  HERC5 downstream primer sequence
ggtgagctttttgcctggggacagaa

SEQ ID NO: 9 - DNA, 24 bp, Artificial Sequence
  HERC6 upstream primer sequence
cactaccactccctggcattatca

SEQ ID NO: 10 - DNA, 26 bp, Artificial Sequence
  HERC6 downstream primer sequence
gccaagtgttttcgtggggaaagaac

SEQ ID NO: 11 - DNA, 24 bp, Artificial Sequence
  IFI27 upstream primer sequence
tcagcttcacattctcaggaactc

SEQ ID NO: 12 - DNA, 26 bp, Artificial Sequence
  IFI27 downstream primer sequence
tctggctgaagttgaggatctcttac

SEQ ID NO: 13 - DNA, 24 bp, Artificial Sequence
  IFI35 upstream primer sequence
gagacccatgtcagccccactgga

SEQ ID NO: 14 - DNA, 26 bp, Artificial Sequence
  IFI35 downstream primer sequence
gccgccctccacgcccttcaggagga

SEQ ID NO: 15 - DNA, 24 bp, Artificial Sequence
  IFI44 upstream primer sequence
acttcttagaggatttgccttttg

SEQ ID NO: 16 - DNA, 26 bp, Artificial Sequence
  IFI44 downstream primer sequence
gaaattatcaactgtgcacaaggaaa

SEQ ID NO: 17 - DNA, 24 bp, Artificial Sequence
  IFI44L upstream primer sequence
tttttagaagatttgcctcttgag

SEQ ID NO: 18 - DNA, 26 bp, Artificial Sequence
  IFI44L downstream primer sequence
tggtgcaattgagagagcgttacagc

SEQ ID NO: 19 - DNA, 24 bp, Artificial Sequence
  IFI6 upstream primer sequence
ctagtggccacgctgcagagcctc

SEQ ID NO: 20 - DNA, 26 bp, Artificial Sequence
  IFI6 downstream primer sequence
gcagcagcgtcgtcataggtaatatt

SEQ ID NO: 21 - DNA, 24 bp, Artificial Sequence
  IFIH1 upstream primer sequence
gaggaactgttgacaattgaagac

SEQ ID NO: 22 - DNA, 26 bp, Artificial Sequence
  IFIH1 downstream primer sequence
gaaaacaatggaaatgaatcaggtgt

SEQ ID NO: 23 - DNA, 24 bp, Artificial Sequence
  IFIT1 upstream primer sequence
cagaacggctgcctaatttacagc

SEQ ID NO: 24 - DNA, 26 bp, Artificial Sequence
  IFIT1 downstream primer sequence
atcaggtcaaggatagtctggagcaa

SEQ ID NO: 25 - DNA, 24 bp, Artificial Sequence
  IFIT3 upstream primer sequence
cagcagagacacagagggcagtca

SEQ ID NO: 26 - DNA, 26 bp, Artificial Sequence
  IFIT3 downstream primer sequence
ggtcaccaagaattccctggagaaaa

SEQ ID NO: 27 - DNA, 24 bp, Artificial Sequence
  IFIT5 upstream primer sequence
tgatggacatgtgaaaagtaagta

SEQ ID NO: 28 - DNA, 26 bp, Artificial Sequence
  IFIT5 downstream primer sequence
attaggctaataggcttaacccagga

SEQ ID NO: 29 - DNA, 24 bp, Artificial Sequence
  IFITM1 upstream primer sequence
ggtctcactgagcaccgtcccagc

SEQ ID NO: 30 - DNA, 26 bp, Artificial Sequence
  IFITM1 downstream primer sequence
agcggcccttcgctccacgcagaaaa

SEQ ID NO: 31 - DNA, 24 bp, Artificial Sequence
  ISG15 upstream primer sequence
gccgtggcccacagcccacagccc

SEQ ID NO: 32 - DNA, 26 bp, Artificial Sequence
  ISG15 downstream primer sequence
ggctgggacctgacggtgaagatgct

SEQ ID NO: 33 - DNA, 24 bp, Artificial Sequence
  MX1 upstream primer sequence
tgctactgtggcccagaaaaatcc

SEQ ID NO: 34 - DNA, 26 bp, Artificial Sequence
  MX1 downstream primer sequence
ctgagaacaacctgtgcagccagtat

SEQ ID NO: 35 - DNA, 24 bp, Artificial Sequence
  MX2 upstream primer sequence
gaaataggcatccacctgaatgcc

SEQ ID NO: 36 - DNA, 26 bp, Artificial Sequence
  MX2 downstream primer sequence
gtctcgccaaccagatcccatttata

SEQ ID NO: 37 - DNA, 24 bp, Artificial Sequence
  OAS1 upstream primer sequence
gctcctaccctgtgtgtgtgtcca

SEQ ID NO: 38 - DNA, 26 bp, Artificial Sequence
  OAS1 downstream primer sequence
gggtggctcctcaggcaagggcacca

SEQ ID NO: 39 - DNA, 24 bp, Artificial Sequence
  OAS2 upstream primer sequence
ccgaacagttccccctggtgcagg

SEQ ID NO: 40 - DNA, 26 bp, Artificial Sequence
  OAS2 downstream primer sequence
gctcctatggacggaaaacagtctta

SEQ ID NO: 41 - DNA, 24 bp, Artificial Sequence
  OAS3 upstream primer sequence
cccgcgggtgctgaaaactgtcaa

SEQ ID NO: 42 - DNA, 26 bp, Artificial Sequence
  OAS3 downstream primer sequence
ggcacagctctcaagggtggctgtga

SEQ ID NO: 43 - DNA, 24 bp, Artificial Sequence
  OASL upstream primer sequence
tgtgcgggtgctgaaggtagtcaa

SEQ ID NO: 44 - DNA, 26 bp, Artificial Sequence
  OASL downstream primer sequence
cgggaatggcacggttctcaggagca

SEQ ID NO: 45 - DNA, 24 bp, Artificial Sequence
  PLSCR1 upstream primer sequence
tcctcagtatccaccgacagcatt

SEQ ID NO: 46 - DNA, 26 bp, Artificial Sequence
  PLSCR1 downstream primer sequence
ctccaggatatagtggctaccctggg

SEQ ID NO: 47 - DNA, 24 bp, Artificial Sequence
  RSAD2 upstream primer sequence
agcaaagagaggattgcttttgct

SEQ ID NO: 48 - DNA, 26 bp, Artificial Sequence
  RSAD2 downstream primer sequence
ggagaagatcaacttttcaggtggag

SEQ ID NO: 49 - DNA, 24 bp, Artificial Sequence
  RTP4 upstream primer sequence
atttctggacttgggagcagacat

SEQ ID NO: 50 - DNA, 26 bp, Artificial Sequence
  RTP4 downstream primer sequence
gaactaatccaagaggcaaaaccccg

SEQ ID NO: 51 - DNA, 24 bp, Artificial Sequence
  SAMD9 upstream primer sequence
tttccccacatatatgtaaaagaa

SEQ ID NO: 52 - DNA, 26 bp, Artificial Sequence
  SAMD9 downstream primer sequence
ctcatggttgcagtgggtgaataggt

SEQ ID NO: 53 - DNA, 24 bp, Artificial Sequence
  SP110 upstream primer sequence
gacaactccatcatcactaagaga

SEQ ID NO: 54 - DNA, 26 bp, Artificial Sequence
  SP110 downstream primer sequence
ctggaagcctgtagaaatttgatccc

SEQ ID NO: 55 - DNA, 24 bp, Artificial Sequence
  USP18 upstream primer sequence
cccagggcctgggactaccctcat

SEQ ID NO: 56 - DNA, 26 bp, Artificial Sequence
  USP18 downstream primer sequence
caacattggacagacctgctgcctta

SEQ ID NO: 57 - DNA, 24 bp, Artificial Sequence
  XAF1 upstream primer sequence
aacatggaaggagacttctcggtg

SEQ ID NO: 58 - DNA, 26 bp, Artificial Sequence
  XAF1 downstream primer sequence
tgccaacttcaccctccatgaggctt

SEQ ID NO: 59 - DNA, 25 bp, Artificial Sequence
  GAPDH upstream primer sequence
tgaggtccaccaccctgttgctgta

SEQ ID NO: 60 - DNA, 25 bp, Artificial Sequence
  GAPDH downstream primer sequence
atgagcttgacaaagtggtcgttga

SEQ ID NO: 61 - DNA, 25 bp, Artificial Sequence
  GUSB upstream primer sequence
attgaagctggagggaactggcatg

SEQ ID NO: 62 - DNA, 25 bp, Artificial Sequence
  GUSB downstream primer sequence
gcggccgccggtaccactgctcctc

SEQ ID NO: 63 - DNA, 25 bp, Artificial Sequence
  MRPL19 upstream primer sequence
ctgttcttccccttcgaggaatgaa

SEQ ID NO: 64 - DNA, 25 bp, Artificial Sequence
  MRPL19 downstream primer sequence
tccacggggcggtgcttgtccacga

SEQ ID NO: 65 - DNA, 25 bp, Artificial Sequence
  PSMC4 upstream primer sequence
tctggggccgggacacggacagtgc

SEQ ID NO: 66 - DNA, 25 bp, Artificial Sequence
  PSMC4 downstream primer sequence
cttctccaccaagatgcctatctcc

SEQ ID NO: 67 - DNA, 25 bp, Artificial Sequence
  SF3A1 upstream primer sequence
gaatcctcctttgaagatgcttctt

SEQ ID NO: 68 - DNA, 25 bp, Artificial Sequence
  SF3A1 downstream primer sequence
ggctgtttgggctccgtgggcacgg

SEQ ID NO: 69 - DNA, 25 bp, Artificial Sequence
  TFRC upstream primer sequence
cttttggagatacgtagggagagag

SEQ ID NO: 70 - DNA, 25 bp, Artificial Sequence
  TFRC downstream primer sequence
cacgatcattgagtttcttcatgac

SEQ ID NO: 71 - DNA, 22 bp, Artificial Sequence
  SAMD9 forward primer sequence
gggcacctactctacaattctg

SEQ ID NO: 72 - DNA, 21 bp, Artificial Sequence
  SAMD9 reverse primer sequence
ccagtgagaactctgcaatca

SEQ ID NO: 73 - DNA, 22 bp, Artificial Sequence
  RTP4 forward primer sequence
cccaatatgagatgcctgagtt

SEQ ID NO: 74 - DNA, 20 bp, Artificial Sequence
  RTP4 reverse primer sequence
gagacttcctcgtgccattt

SEQ ID NO: 75 - DNA, 22 bp, Artificial Sequence
  OASL forward primer sequence
gccatgtactccagaactcatc

SEQ ID NO: 76 - DNA, 22 bp, Artificial Sequence
  OASL reverse primer sequence
ggcctgggataactcattgtaa

SEQ ID NO: 77 - DNA, 22 bp, Artificial Sequence
  OAS2 forward primer sequence
catagaccctcaggagagaaga

SEQ ID NO: 78 - DNA, 22 bp, Artificial Sequence
  OAS2 reverse primer sequence
tccaaagacaatcagggtatgg

SEQ ID NO: 79 - DNA, 20 bp, Artificial Sequence
  IFIT3 forward primer sequence
agtggctcatgcctgtaatc

SEQ ID NO: 80 - DNA, 22 bp, Artificial Sequence
  IFIT3 reverse primer sequence
agacgggatttcactgtgttag

SEQ ID NO: 81 - DNA, 21 bp, Artificial Sequence
  IFI35 forward primer sequence
gagcctcctgaggtgtatttc

SEQ ID NO: 82 - DNA, 22 bp, Artificial Sequence
  IFI35 reverse primer sequence
cacagcaactgaactcttgttt

SEQ ID NO: 83 - DNA, 21 bp, Artificial Sequence
  ACTB forward primer sequence
ggatcagcaagcaggagtatg

SEQ ID NO: 84 - DNA, 22 bp, Artificial Sequence
  ACTB reverse primer sequence
agaaagggtgtaacgcaactaa

SEQ ID NO: 85 - DNA, 23 bp, Artificial Sequence
  GAPDH forward primer sequence
ggtgtgaaccatgagaagtatga

SEQ ID NO: 86 - DNA, 22 bp, Artificial Sequence
  GAPDH reverse primer sequence
gagtccttccacgataccaaag

SEQ ID NO: 87 - DNA, 22 bp, Artificial Sequence
  RPLP0 forward primer sequence
ggagaaactgctgcctcatatc

SEQ ID NO: 88 - DNA, 19 bp, Artificial Sequence
  RPLP0 reverse primer sequence
cagcagctggcaccttatt

SEQ ID NO: 89 - DNA, 26 bp, Artificial Sequence
  SAMD9 probe sequence
aatgtgggaactactgtggagtacgc

SEQ ID NO: 90 - DNA, 24 bp, Artificial Sequence
  RTP4 probe sequence
tctgagcaacctggtgcagcatat

SEQ ID NO: 91 - DNA, 24 bp, Artificial Sequence
  OASL probe sequence
aactggtgaagacctgggaccttc

SEQ ID NO: 92 - DNA, 25 bp, Artificial Sequence
  OAS2 probe sequence
tggactcgctctcttctctggaact

SEQ ID NO: 93 - DNA, 24 bp, Artificial Sequence
  IFIT3 probe sequence
caaactccagacctcgtgatccgc

SEQ ID NO: 94 - DNA, 24 bp, Artificial Sequence
  IFI35 probe sequence
caaagggctgtggtctctctcagc

SEQ ID NO: 95 - DNA, 24 bp, Artificial Sequence
  ACTB probe sequence
tcgtccaccgcaaatgcttctagg

SEQ ID NO: 96 - DNA, 23 bp, Artificial Sequence
  GAPDH probe sequence
ggtgtgaaccatgagaagtatga

SEQ ID NO: 97 - DNA, 24 bp, Artificial Sequence
  RPLP0 probe sequence
tggtgaacacaaagcccacattcc